(19) United States
(12) Patent Application Publication
(10) Pub. No.: US 2026/0308736 A1
(43) Pub. Date: Oct. 8, 2026

(54) METAL ALLOY THAT INCLUDES RADIOACTIVE RHENIUM

(71) Applicant: MiRus LLC, Marietta, GA (US)

(72) Inventor: Noah Roth, Marietta, GA (US)

(21) Appl. No.: 19/571,710

(22) Filed: Mar. 19, 2026

Related U.S. Application Data
(60) Provisional application No. 63/783,022, filed on Apr. 3, 2025.

(51) Int. Cl. C22C 43/00; A61N 5/10

(52) U.S. Cl. CPC C22C 43/00; A61N 5/1001

(57) ABSTRACT

A medical device that is at least partially formed of a metal alloy that includes rhenium; and wherein 0.5-100% of the rhenium in the metal alloy is formed of radioactive isotopes of rhenium.

### REFERENCED APPLICATION

The present application claims priority to United States Applicant Serial No. 63/783,022 filed April 3, 2025, which is fully incorporated herein.

### TECHNICAL FIELD

The present disclosure relates generally to a metal alloy that includes radioactive rhenium, and particularly to a medical device that is partially or fully formed of a metal alloy that includes radioactive rhenium.

### BACKGROUND OF DISCLOSURE

Many cardiovascular devices such as stents, expandable heart valves, and the like are inserted into a patient via the vascular system of a patient. Likewise, many orthopedic devices such as spinal implants, bone plates, bone implants, and the like are implanted in a patient. For some patients, radiation treatment is required at or near the locations that the cardiovascular devices or orthopedic devices are implanted. In such situations, a radioactive medication can be applied locally or system wide to treat the patient, and/or the patient can be subjected to radiation treatment.

In view of the current state of medical devices, there is a need for a medical device to provide localized radiation treatment after implantation.

### SUMMARY OF DISCLOSURE

The present disclosure is directed to a metal alloy that includes radioactive rhenium, particularly to a medical device that is partially or fully formed of a metal alloy that includes radioactive rhenium, and more particularly to a medical device that is at least partially or fully made of a metal alloy that includes rhenium (e.g., 5-100% and all values and ranges therebetween of the medical device is formed of a metal alloy that includes rhenium), and wherein the rhenium in the metal alloy partially or fully includes a radioactive isotope of rhenium. In one non-limiting embodiment, 5-100% (and all values and ranges therebetween) of the rhenium in the metal alloy that include rhenium is a radioactive isotope of rhenium. The use of radioactive isotopes of rhenium as part of a metal alloy that partially or fully forms the medical device can be used to provide localized radiation at a treatment site after implantation of the medical device in a patient. For example, when the medical device is a bone plate that is partially or fully formed of a metal alloy that includes rhenium, and wherein a portion or all of the rhenium in the metal alloy is a radioactive isotope of rhenium, the implanted bone plate can be used to a) facilitate in the treatment of bone cancer that is located at or near the implanted bone plate when implanted at the treatment site, and/or b) facilitate in the treatment of cancer and/or the prevention of the redevelopment of cancer at or near a location that the cancerous cells currently exist and/or were removed from the patient at or near a treatment site. As can be appreciated, localize injections of radioactive isotopes of rhenium can also be injected at or near a location of implantation of the bone plate to a) facilitate in the treatment of bone cancer that is located at or near the implanted bone plate when implanted at the treatment site, and/or b) facilitate in the treatment of cancer and/or the prevention of the redevelopment of cancer at or near a location that the cancerous cells currently exist and/or were removed from the patient at or near a treatment site. As can be appreciated, localize injections of radioactive isotopes of rhenium at a treatment site can be used independently of implantation of a medical device that does or does not include radioactive isotopes of rhenium (e.g., Brachytherapy that includes the use of wires, seeds, rods, glass/ceramic beads, etc. that filled with radioactive materials and injected into a patient, etc.). As can also be appreciated, the non-limiting example above regarding a medical device in the form of a bone plate is non-limiting, and other medical devices that include radioactive isotopes of rhenium can be used to facilitate in the treatment or prevention of cancer. The medical devices in accordance with the present disclosure can include an orthopedic device; PFO (patent foramen ovale) device; stent (e.g., stent for used in aortic, iliac, subclavian, carotid, femoral artery, tibial, intracranial arteries, etc.); valve (e.g., heart valve, TAVR valve, mitral valve replacement, tricuspid valve replacement, pulmonary valve replacement, etc.); aneurysm exclusion devices (e.g., devices for aneurysm for use in aorta, iliac, intracranial arteries, etc.); LAA (left atrial appendage) closure devices; anchoring devices for valves (e.g., anchoring devices for heart valve, TAVR valve, aortic valve, mitral valve, tricuspid valve, pulmonary valve, etc.); valve frames; occluders (e.g., occluders for patent foramen ovale, ventricular septal defect, left atrial appendage, etc.); spinal implant; frame and other structures for use with a spinal implant; vascular implant; graft; staple; pacemaker; dental implant; dental crown; dental braces; wire used in medical procedures; bone implant; artificial disk; artificial spinal disk; prosthetic implant or device to repair, replace and/or support a bone (e.g., acromion, atlas, axis, calcaneus, carpus, clavicle, coccyx, epicondyle, epitrochlea, femur, fibula, frontal bone, greater trochanter, humerus, ilium, ischium, mandible, maxilla, metacarpus, metatarsus, occipital bone, olecranon, parietal bone, patella, phalanx, radius, ribs, sacrum, scapula, sternum, talus, tarsus, temporal bone, tibia, ulna, zygomatic bone, etc.) and/or cartilage; bone plate; orthopedic nail; orthopedic rod; orthopedic screw; orthopedic post; orthopedic cage; orthopedic hinge; orthopedic joint system; orthopedic anchor; orthopedic spacer; orthopedic disk; orthopedic ball; orthopedic locking connector other structural assembly that is used in a body to support a structure, mount a structure, and/or repair a structure in a body such as, but not limited to, a human body, animal body, etc. In one non-limiting embodiment, the medical device includes an orthopedic device (e.g., prosthetic implant or device to repair, replace and/or support a bone (e.g., acromion, atlas, axis, calcaneus, carpus, clavicle, coccyx, epicondyle, epitrochlea, femur, fibula, frontal bone, greater trochanter, humerus, ilium, ischium, mandible, maxilla, metacarpus, metatarsus, occipital bone, olecranon, parietal bone, patella, phalanx, radius, ribs, sacrum, scapula, sternum, talus, tarsus, temporal bone, tibia, ulna, zygomatic bone, etc.) orthopedic nail, orthopedic rod, orthopedic screw, orthopedic post, or spinal implant, etc.).

In one non-limiting aspect of the present disclosure, there is provided a rhenium containing metal alloy wherein the rhenium content of the metal alloy includes 25-100% (and all values and ranges therebetween) of a radioactive isotope of rhenium. Non-limiting radioactive isotopes of rhenium that can be used in the metal alloy include Re-173, Re-174, Re-175, Re-176, Re-177, Re-178, Re-179, Re-180, Re-181, Re-182, Re-183, Re-184, Re-186, Re-188, Re-189, Re-190 and/or Re-191. In one non-limiting configuration, the radioactive isotopes of rhenium that can be used in the metal alloy include Re-181, Re-182, Re-183, Re-184, Re-186, Re-188 and/or Re-189. In another non-limiting configuration, the radioactive isotopes of rhenium that can be used in the metal alloy include Re-186, Re-188 and/or Re-189. In another non-limiting configuration, 80-100% (and all values and ranges therebetween) of the radioactive isotopes of rhenium used in the metal alloy are Re-188. The formation of the radioactive isotopes of rhenium are non-limiting. In one non-limiting method, the metal alloy in a form prior to being partially or fully formed into the medical device (e.g., rod of metal alloy, plate of metal alloy, pedicle screw, stent, frame of a medical device, etc.), and/or after the metal alloy has been partially or fully formed into the medical device can placed in a reactor and exposed to neutron irradiation. For example, Re-188 can be formed by neutron irradiation of Re-187 and/or RE-185. Re-188 can also be formed in an isotope generator by radioactive decay of W-188.

In another non-limiting aspect of the present disclosure, there is provided a rhenium containing metal alloy wherein the rhenium content of the metal alloy includes 25-100% of a radioactive isotope of rhenium, and the rhenium content of the metal alloy is at least 5 awt.% (atomic percent) (e.g., 5-90 awt.% and all values and ranges therebetween). As used herein, atomic weight percent (awt.%) or atomic percentage (at.%) or atomic percent (at.%) are used interchangeably. The abbreviations at.% and awt.% are used interchangeably. As defined herein, the weight percentage (wt.%) of an element is the weight of that element measured in the sample divided by the weight of all elements in the sample multiplied by 100. The atomic percentage, atomic percent or atomic weight percent (awt.% or at.%) is the number of atoms of that element, at that weight percentage, divided by the total number of atoms in the sample multiplied by 100. The use of the terms weight percentage (wt.%) and atomic percentage or atomic weight percentage or atomic percent (awt.% or at.%) are two ways of referring to metallic alloy and its constituents. In another non-limiting embodiment, the amount of rhenium that is included in the material that is used to partially or fully form the medical device is at least 15 awt.% (atomic weight percent) (e.g., 15-90 wt.% and all values and ranges therebetween). In another non-limiting embodiment, the amount of rhenium that is included in the material that is used to partially or fully form the medical device is at least 5 wt.% (weight percent) (e.g., 5-90 wt.% and all values and ranges therebetween). In another non-limiting embodiment, the medical device that is formed of 5-100 wt.% of a metal alloy that includes rhenium includes at least a sufficient amount of rhenium to create a “rhenium effect” in the metal alloy. As defined herein, a “rhenium effect” is a) an increase of at least 10% in ductility of the metal alloy caused by the addition of rhenium to the metal alloy as compared to the same metal alloy that is absent rhenium, and/or b) an increase of at least 10% in tensile strength of the metal alloy caused by the addition of rhenium to the metal alloy as compared to the same metal alloy that is absent rhenium. In some metal alloys, the rhenium effect occurs when the atomic weight of rhenium in the metal alloy is at least 15 awt.%; however, for other metal alloys, the rhenium effect can occur when the metal alloy includes less than or more than 15 awt.% rhenium.

In another non-limiting aspect of the present disclosure, there is provided a rhenium containing metal alloy that optionally includes molybdenum, and wherein 0-100% (and all values and ranges therebetween) of the molybdenum in the metal alloy is a radioactive isotope of molybdenum. Non-limiting radioactive isotopes of molybdenum that can be used in the metal alloy include Mo-86, Mo-87, Mo-88, Mo-89, Mo-90, Mo-91, Mo-99, Mo-101, Mo-102, Mo-103, Mo-104, Mo-105, Mo-106 and/or Mo-107. In one non-limiting configuration, the radioactive isotopes of molybdenum that can be used in the metal alloy include Mo-99.

In accordance with another and/or alternative aspect of the present disclosure, the metal alloy includes at least 5 awt.% rhenium (e.g., 5-90 wt.% and all values and ranges therebetween), and at least 0.1 wt.% (e.g., 0.1 wt.% to 96 wt.% and all values and ranges therebetween) of one or more metal additives selected from aluminum, boron, beryllium, bismuth, cadmium, calcium, carbon, cerium, chromium, cobalt, copper, gallium, gold, hafnium, iridium, iron, lanthanum, lithium, magnesium, manganese, molybdenum, nickel, niobium, osmium, palladium, platinum, rare earth metals, rhodium, ruthenium, silicon, scandium, silver, silicon, tantalum, technetium, tin, titanium, tungsten, vanadium, yttrium, zinc, and/or zirconium, and the metal alloy optionally includes 0-2 wt.% (and all values and ranges therebetween) of a combination of metals other than rhenium and metal additives, oxygen, phosphorous, sulfur, hydrogen and/or nitrogen, and wherein 0.5-100% (and all values and ranges therebetween) of the rhenium content of the metal alloy is a radioactive isotope of rhenium.

In accordance with another and/or alternative aspect of the present disclosure, the metal alloy includes rhenium in a sufficient amount to create a rhenium effect in the metal alloy, and the metal alloy is a refractory metal alloy, and wherein 0.5-100% (and all values and ranges therebetween) of the rhenium content of the metal alloy is a radioactive isotope of rhenium. As defined herein, a refractory metal alloy is a metal alloy that includes at least 02 wt.% of one or more of molybdenum, rhenium, hafnium, niobium, tantalum or tungsten. Non-limiting types of refractory metal alloys include MoRe alloy (e.g., 40-60 wt.% Mo and 40-60% Re and all values and ranges therebetween; 52.5 wt.% Mo and 47.5 wt.% Re, etc.), ReW alloy, MoReCr alloy, MoReTa alloy, MoReTi alloy, WCu alloy, ReCr, molybdenum alloy, rhenium alloy, tungsten alloy, tantalum alloy, niobium alloy, hafnium alloy, etc.

In accordance with another and/or alternative aspect of the present disclosure, the metal alloy is a stainless steel alloy that has been modified to include at least 5 awt.% rhenium, and wherein 0.5-100% (and all values and ranges therebetween) of the rhenium content of the metal alloy is a radioactive isotope of rhenium, and wherein the metal alloy optionally includes 0-2 wt.% (and all values and ranges therebetween) of a combination of metals other than rhenium and the metal components that form the stainless steel alloy, carbon, oxygen, phosphorous, sulfur, hydrogen and/or nitrogen. As defined herein, a stainless-steel alloy (SS alloy) includes at least 50 wt.% iron, and one or more of a) 9-28 wt.% chromium, b) 0-35 wt.% nickel, c) 0-7 wt.% molybdenum, d) 0-16 wt.% manganese, e) 0-4 wt.% silicon, f) 0-0.3 wt.% carbon, h) 0.01-1 wt.% cobalt, h) 0-5 wt.% titanium, i) 0-10 wt.% niobium, j) 0-5 wt.% copper, k) 0-4 wt.% aluminum, l) 0-10 wt. % tantalum, m) 0-1 wt.% selenium, n) 0-2 wt.% vanadium, and o) 0-2 wt.% tungsten. A 316L alloy that falls within a stainless-steel alloy includes 17-19 wt.% chromium, 13-15 wt.% nickel, 2-4 wt.% molybdenum, 2 wt.% max manganese, 0.75 wt.% max silicon, 0.03 wt.% max carbon, balance iron.

In accordance with another and/or alternative aspect of the present disclosure, the metal alloy is a cobalt-chromium alloy that has been modified to include at least 5 awt.% rhenium, and wherein 0.5-100% (and all values and ranges therebetween) of the rhenium content of the metal alloy is a radioactive isotope of rhenium, and wherein the metal alloy optionally includes 0-2 wt.% (and all values and ranges therebetween) of a combination of metals other than rhenium and the metal components that form the cobalt-chromium alloy, carbon, oxygen, phosphorous, sulfur, hydrogen and/or nitrogen. As defined herein, a cobalt-chromium alloy (CoCr alloy) includes 30-68 wt.% cobalt, 12-32 wt.% chromium, and optionally one or more of 0.01-38 wt.% nickel, 0.1-30 wt.% molybdenum, 0-18 wt.% iron, 0-5 wt.% titanium, 0-2 wt.% manganese, 0-0.5 wt.% lanthanum, 0-0.25 wt.% carbon, 0-18 wt.% tungsten, 0-2 wt.% silicon, 0-2 wt.% aluminum, 0-2 wt.% niobium, 0-20 wt.% iron, 0-0.5 wt.% boron, 0-0.15 wt.% silver, and 0-2 wt.% titanium. As a MP35N alloy that falls within a CoCr alloy includes 18-22 wt.% chromium, 32-38 wt.% nickel, 8-12 wt.% molybdenum, 0-2 wt.% iron, 0-0.5 wt.% silicon, 0-0.5 wt.% manganese, 0-0.2 wt.% carbon, 0-2 wt.% titanium, 0-0.1 wt.%, 0-0.1 wt.% boron, 0-0.15 wt.% silver, and balance cobalt. As defined herein, a Phynox and Elgiloy alloy that falls within a CoCr alloy includes 38-42 wt.% cobalt, 18-22 wt.% chromium, 14-18 wt.% iron, 13-17 wt.% nickel, 6-8 wt.% molybdenum. As defined herein, a L605 alloy that falls within a CoCr alloy includes 18-22 wt.% chromium, 14-16 wt.% tungsten, 9-11 wt.% nickel, balance cobalt.

In accordance with another and/or alternative aspect of the present disclosure, the metal alloy is a TiAlV alloy that has been modified to include at least 5 awt.% rhenium, and wherein 0.5-100% (and all values and ranges therebetween) of the rhenium content of the metal alloy is a radioactive isotope of rhenium, and wherein the metal alloy optionally includes 0-2 wt.% (and all values and ranges therebetween) of a combination of metals other than rhenium and the metal components that form the TiAlV alloy, carbon, oxygen, phosphorous, sulfur, hydrogen and/or nitrogen. As defined herein, a titanium-aluminum-vanadium alloy (TiAlV alloy) includes 2–8 wt.% aluminum, 0.01-16 wt.% vanadium, 80-93 wt.% titanium, and optionally one or more of 0-1 wt.% iron, 0-0.2 wt.% carbon, 0-20 wt.% chromium, 0-16 wt.% molybdenum, 0-2 wt.% nickel, 0-12 wt.% tin, 0-6 wt.% zirconium, 0-2 wt.% tantalum, 0-4 wt.% niobium, 0-1 wt.% silicon, 0-3 wt.% iron, and 0-0.5 wt.% yttrium. A Ti-6Al-4V alloy that falls with a TiAlV alloy includes incudes 3.5-4.5 wt.% vanadium, 5.5-6.75 wt.% aluminum, 0.3 wt.% max iron, 0.08 wt.% max carbon, 0.05 wt.% max yttrium, balance titanium.

In accordance with another and/or alternative aspect of the present disclosure, the metal alloy is an aluminum alloy that has been modified to include at least 5 awt.% rhenium, and wherein 0.5-100% (and all values and ranges therebetween) of the rhenium content of the metal alloy is a radioactive isotope of rhenium, and wherein the metal alloy optionally includes 0-2 wt.% (and all values and ranges therebetween) of a combination of metals other than rhenium and the metal additives that form the aluminum alloy, carbon, oxygen, phosphorous, sulfur, hydrogen and/or nitrogen. As defined herein, an aluminum alloy includes 80-99 wt.% aluminum, and optionally one or more 0-12 wt.% silicon, 0-5 wt.% magnesium, 0-1 wt.% manganese, 0-0.5 wt.% scandium, 0-0.5 wt.% beryllium, 0-0.5 wt.% yttrium, 0-0.5 wt.% cerium, 0-0.5 wt.% chromium, 0-3 wt.% iron, 0-9 wt.% zinc, 0-0.5 wt.% titanium, 0-3 wt.% lithium, 0-0.5 wt.% silver, 0-0.5 wt.% calcium, 0-0.5 wt.% zirconium, 0-1 wt.% lead, 0-0.5 wt.% cadmium, 0-0.05 wt.% bismuth, 0-1 wt.% nickel, 0-0.2 wt.% vanadium, 0-0.1 wt.% gallium, and 0-7 wt.% copper.

In accordance with another and/or alternative aspect of the present disclosure, the metal alloy is a nickel alloy that has been modified to include at least 5 awt.% rhenium, and wherein 0.5-100% (and all values and ranges therebetween) of the rhenium content of the metal alloy is a radioactive isotope of rhenium, and wherein the metal alloy optionally includes 0-2 wt.% (and all values and ranges therebetween) of a combination of metals other than rhenium and the metal components that form the nickel alloy, carbon, oxygen, phosphorous, sulfur, hydrogen and/or nitrogen. As defined herein, a nickel alloy includes 30-98 wt.% nickel, and optionally one or more 5-25 wt.% chromium, 0-65 wt.% iron, 0-30 wt.% molybdenum, 0-32 wt.% copper, 0-32 wt.% cobalt, 2-2 wt.% aluminum, 0-6 wt.% tantalum, 0-15 wt.% tungsten, 0-5 wt.% titanium, 0-6 wt.% niobium, 0-3 wt.% silicon.

In accordance with another and/or alternative aspect of the present disclosure, the metal alloy is a titanium alloy that has been modified to include at least 5 awt.% rhenium, and wherein 0.5-100% (and all values and ranges therebetween) of the rhenium content of the metal alloy is a radioactive isotope of rhenium, and wherein the metal alloy optionally includes 0-2 wt.% (and all values and ranges therebetween) of a combination of metals other than rhenium and the metal components that form the titanium alloy, carbon, oxygen, phosphorous, sulfur, hydrogen and/or nitrogen. As defined herein, a titanium alloy includes 80-99 wt.% titanium, and optionally one of more of 0-6 wt.% aluminum, 0-3 wt.% tin, 0-1 wt.% palladium, 0-8 wt.% vanadium, 0-15 wt.% molybdenum, 0-1 wt.% nickel, 0-0.3 wt.% ruthenium, 0-6 wt.% chromium, 0-4 wt.% zirconium, 0-4 wt.% niobium, 0-1 wt.% silicon, 0.0.5 wt.% cobalt, 0-2 wt.% iron.

In accordance with another and/or alternative aspect of the present disclosure, the metal alloy is a tungsten alloy that has been modified to include at least 5 awt.% rhenium, and wherein 0.5-100% (and all values and ranges therebetween) of the rhenium content of the metal alloy is a radioactive isotope of rhenium, and wherein the metal alloy optionally includes 0-2 wt.% (and all values and ranges therebetween) of a combination of metals other than rhenium and the metal components that form the tungsten alloy, carbon, oxygen, phosphorous, sulfur, hydrogen and/or nitrogen. As defined herein, a tungsten alloy includes 85-98 wt.% tungsten, and optionally one or more of 0-8 wt.% nickel, 0-5 wt.% copper, 0-5 wt.% molybdenum, 0-4 wt.% iron.

In accordance with another and/or alternative aspect of the present disclosure, the metal alloy is a molybdenum alloy that has been modified to include at least 5 awt.% rhenium, and wherein 0.5-100% (and all values and ranges therebetween) of the rhenium content of the metal alloy is a radioactive isotope of rhenium, and wherein the metal alloy optionally includes 0-2 wt.% (and all values and ranges therebetween) of a combination of metals other than rhenium and the metal components that form the molybdenum alloy, carbon, oxygen, phosphorous, sulfur, hydrogen and/or nitrogen. As defined herein, molybdenum alloy includes 90-99.5 wt.% molybdenum, 0-1 wt.% nickel, 0-1 wt.% titanium, 0-1 wt.% zirconium, 0-30 wt.% tungsten, 0-2 wt.% hafnium, 0-2 wt.% lanthanum.

In accordance with another and/or alternative aspect of the present disclosure, the metal alloy is a copper alloy that has been modified to include at least 5 awt.% rhenium, and wherein 0.5-100% (and all values and ranges therebetween) of the rhenium content of the metal alloy is a radioactive isotope of rhenium, and wherein the metal alloy optionally includes 0-2 wt.% (and all values and ranges therebetween) of a combination of metals other than rhenium and the metal components that form the copper alloy, carbon, oxygen, phosphorous, sulfur, hydrogen and/or nitrogen. As defined herein, a copper alloy includes 55-95 wt.% copper, and optionally one or more of 0-40 wt.% zinc, 0-10 wt.% tin, 0-10 wt.% lead, 0-1 wt.% iron, 0-5 wt.% silicon, 0-12 wt.% manganese, 0-12 wt.% aluminum, 0-3 wt.% beryllium, 0-1 wt.% cobalt, 0-20 wt.% nickel.

In accordance with another and/or alternative aspect of the present disclosure, the metal alloy is a beryllium-copper alloy that has been modified to include at least 5 awt.% rhenium, and wherein 0.5-100% (and all values and ranges therebetween) of the rhenium content of the metal alloy is a radioactive isotope of rhenium, and wherein the metal alloy optionally includes 0-2 wt.% (and all values and ranges therebetween) of a combination of metals other than rhenium and the metal additives that form the beryllium-copper alloy, carbon, oxygen, phosphorous, sulfur, hydrogen and/or nitrogen. As defined herein, a beryllium-copper alloy includes 95-98.5 wt.% copper, 1-4 wt.% beryllium, 0-1 wt.% cobalt, and 0-0.5 wt.% silicon.

In accordance with another and/or alternative aspect of the present disclosure, the metal alloy is a titanium-nickel alloy that has been modified to include at least 5 awt.% rhenium, and wherein 0.5-100% (and all values and ranges therebetween) of the rhenium content of the metal alloy is a radioactive isotope of rhenium, and wherein the metal alloy optionally includes 0-2 wt.% (and all values and ranges therebetween) of a combination of metals other than rhenium and the metal additives that form the titanium-nickel alloy, carbon, oxygen, phosphorous, sulfur, hydrogen and/or nitrogen. As defined herein, a titanium-nickel alloy (e.g., Nitinol alloy) includes 42-58 wt.% nickel and 42-58 wt.% titanium.

In accordance with another and/or alternative aspect of the present disclosure, the metal alloy includes at least 5 awt.% (e.g., 5-99 awt.% and all values and ranges therebetween) rhenium plus a metal additive that includes at least two metal additives selected from the group of molybdenum, bismuth, chromium, iridium, niobium, tantalum, titanium, yttrium, and zirconium, and the content of the metal alloy that includes other elements and compounds is 0-0.1 wt.%, and wherein 0.5-100% (and all values and ranges therebetween) of the rhenium content of the metal alloy is a radioactive isotope of rhenium, and wherein the metal alloy optionally includes 0-2 wt.% (and all values and ranges therebetween) of a combination of metals other than rhenium and the metal additives, carbon, oxygen, phosphorous, sulfur, hydrogen and/or nitrogen.

In accordance with another and/or alternative aspect of the present disclosure, the metal alloy includes rhenium, chromium and optionally molybdenum. In another non-limiting embodiment, the metal alloy includes at least 35 wt.% (e.g., 35-75 wt.% and all values and ranges therebetween) rhenium and chromium, and wherein 0.5-100% (and all values and ranges therebetween) of the rhenium content of the metal alloy is a radioactive isotope of rhenium. In one non-limiting embodiment, the metal alloy includes at least 35 wt.% rhenium, at least 25 wt.% (e.g., 25-49.9 wt.% and all values and ranges therebetween) chromium, and optionally 0.1-40 wt.% (and all values and ranges therebetween) one or more metal additives selected from aluminum, boron, beryllium, bismuth, cadmium, calcium, cerium, chromium, cobalt, copper, gallium, gold, hafnium, iridium, iron, lanthanum, lithium, magnesium, manganese, molybdenum, nickel, niobium, osmium, palladium, platinum, rare earth metals, rhodium, ruthenium, scandium, silver, silicon, tantalum, technetium, tin, titanium, tungsten, vanadium, yttrium, zinc, and/or zirconium, and wherein 0.5-100% (and all values and ranges therebetween) of the rhenium content of the metal alloy is a radioactive isotope of rhenium, and wherein the metal alloy optionally includes 0-2 wt.% (and all values and ranges therebetween) of a combination of metals other than rhenium, chromium and the metal additives, carbon, oxygen, phosphorous, sulfur, hydrogen and/or nitrogen. In another non-limiting embodiment, the metal alloy includes at least 50 wt.% (e.g., 50-75 wt.% and all values and ranges therebetween) rhenium, at least 25 wt.% chromium (e.g., 25-50 wt.% and all values and ranges therebetween), and optionally 0.1-40 wt.% (and all values and ranges therebetween) of one or more metal additives selected from aluminum, boron, beryllium, bismuth, cadmium, calcium, cerium, cobalt, copper, gallium, gold, hafnium, iridium, iron, lanthanum, lithium, magnesium, manganese, molybdenum, nickel, niobium, osmium, palladium, platinum, rare earth metals, rhodium, ruthenium, scandium, silver, silicon, tantalum, technetium, tin, titanium, tungsten, vanadium, yttrium, zinc, and/or zirconium, and wherein 0.5-100% (and all values and ranges therebetween) of the rhenium content of the metal alloy is a radioactive isotope of rhenium, and wherein the metal alloy optionally includes 0-2 wt.% (and all values and ranges therebetween) of a combination of metals other than rhenium and chromium and the metal additives, carbon, oxygen, phosphorous, sulfur, hydrogen and/or nitrogen, and wherein a combined weight percent of the rhenium and the chromium is 75-100 wt.% (and all values and ranges therebetween) of the metal alloy. In another non-limiting embodiment, the metal alloy includes at least 10 wt.% (e.g., 10-75 wt.% and all values and ranges therebetween) rhenium and at least 2 wt.% chromium (e.g., 2-50 wt.% and all values and ranges therebetween), at least 10 wt.% molybdenum (e.g., 10-75 wt.% and all values and ranges therebetween), and optionally 0.1-40 wt.% (and all values and ranges therebetween) of the metal alloy includes one or more metal additives selected from aluminum, boron, beryllium, bismuth, cadmium, calcium, cerium, cobalt, copper, gallium, gold, hafnium, iridium, iron, lanthanum, lithium, magnesium, manganese, nickel, niobium, osmium, palladium, platinum, rare earth metals, rhodium, ruthenium, scandium, silver, silicon, tantalum, technetium, tin, titanium, tungsten, vanadium, yttrium, zinc, and/or zirconium, and wherein 0.5-100% (and all values and ranges therebetween) of the rhenium content of the metal alloy is a radioactive isotope of rhenium, and wherein the metal alloy optionally includes 0-2 wt.% (and all values and ranges therebetween) of a combination of metals other than rhenium and chromium and molybdenum and the metal additives, carbon, oxygen, phosphorous, sulfur, hydrogen and/or nitrogen, and wherein a combined weight percent of rhenium, molybdenum and chromium is 75-100 wt.% (and all values and ranges therebetween) of the metal alloy.

In accordance with another and/or alternative aspect of the present disclosure, the metal alloy includes a) 15-50 awt.% rhenium (and all values and ranges therebetween), b) 0.5-70 awt.% chromium (and all values and ranges therebetween), and c) 0.1-40 wt.% (and all values and ranges therebetween) of one or more metal additives selected from aluminum, boron, beryllium, bismuth, cadmium, calcium, carbon, cerium, cobalt, copper, gallium, gold, hafnium, iridium, iron, lanthanum, lithium, magnesium, manganese, molybdenum, nickel, niobium, osmium, palladium, platinum, rare earth metals, rhodium, ruthenium, scandium, silver, silicon, tantalum, technetium, tin, titanium, tungsten, vanadium, yttrium, zinc, and zirconium, and wherein 0.5-100% (and all values and ranges therebetween) of the rhenium content of the metal alloy is a radioactive isotope of rhenium, and wherein the metal alloy optionally includes 0-2 wt.% (and all values and ranges therebetween) of a combination of metals other than rhenium and chromium and the metal additives, carbon, oxygen, phosphorous, sulfur, hydrogen and/or nitrogen.

In accordance with another and/or alternative aspect of the present disclosure, the metal alloy includes a) 15-50 awt.% rhenium (and all values and ranges therebetween), b) 0.5-70 awt.% tantalum (and all values and ranges therebetween), and c) 0.1-40 wt.% (and all values and ranges therebetween) of one or more metal additives selected from aluminum, boron, beryllium, bismuth, cadmium, calcium, carbon, cerium, chromium, cobalt, copper, gallium, gold, hafnium, iridium, iron, lanthanum, lithium, magnesium, manganese, molybdenum, nickel, niobium, osmium, palladium, platinum, rare earth metals, rhodium, ruthenium, scandium, silver, silicon, technetium, tin, titanium, tungsten, vanadium, yttrium, zinc, and zirconium, and wherein 0.5-100% (and all values and ranges therebetween) of the rhenium content of the metal alloy is a radioactive isotope of rhenium, and wherein the metal alloy optionally includes 0-2 wt.% (and all values and ranges therebetween) of a combination of metals other than rhenium and tantalum and the metal additives, carbon, oxygen, phosphorous, sulfur, hydrogen and/or nitrogen.

In accordance with another and/or alternative aspect of the present disclosure, the metal alloy includes a) 15-50 awt.% rhenium (and all values and ranges therebetween), b) 0.5-70 awt.% niobium (and all values and ranges therebetween), and c) 0.1-40 wt.% (and all values and ranges therebetween) of one or more metal additives selected from aluminum, boron, beryllium, bismuth, cadmium, calcium, carbon, cerium, chromium, cobalt, copper, gallium, gold, hafnium, iridium, iron, lanthanum, lithium, magnesium, manganese, molybdenum, nickel, osmium, palladium, platinum, rare earth metals, rhodium, ruthenium, scandium, silver, silicon, tantalum, technetium, tin, titanium, tungsten, vanadium, yttrium, zinc, and zirconium, and wherein 0.5-100% (and all values and ranges therebetween) of the rhenium content of the metal alloy is a radioactive isotope of rhenium, and wherein the metal alloy optionally includes 0-2 wt.% (and all values and ranges therebetween) of a combination of metals other than rhenium and niobium and the metal additives, carbon, oxygen, phosphorous, sulfur, hydrogen and/or nitrogen.

In accordance with another and/or alternative aspect of the present disclosure, the metal alloy includes a) 15-50 awt.% rhenium (and all values and ranges therebetween), b) 0.5-70 awt.% titanium (and all values and ranges therebetween), and c) 0.1-40 wt.% (and all values and ranges therebetween) of one or more metal additive selected from aluminum, boron, beryllium, bismuth, cadmium, calcium, carbon, cerium, chromium, cobalt, copper, gallium, gold, hafnium, iridium, iron, lanthanum, lithium, magnesium, manganese, molybdenum, nickel, niobium, osmium, palladium, platinum, rare earth metals, rhodium, ruthenium, scandium, silver, silicon, tantalum, technetium, tin, tungsten, vanadium, yttrium, zinc, and zirconium, and wherein 0.5-100% (and all values and ranges therebetween) of the rhenium content of the metal alloy is a radioactive isotope of rhenium, and wherein the metal alloy optionally includes 0-2 wt.% (and all values and ranges therebetween) of a combination of metals other than rhenium and titanium and the metal additives, carbon, oxygen, phosphorous, sulfur, hydrogen and/or nitrogen.

In accordance with another and/or alternative aspect of the present disclosure, the metal alloy includes a) 15-50 awt.% rhenium (and all values and ranges therebetween), b) 0.5-70 awt.% zirconium (and all values and ranges therebetween), and c) 0.1-40 wt.% (and all values and ranges therebetween) of one or more metal additive selected from aluminum, boron, beryllium, bismuth, cadmium, calcium, carbon, cerium, chromium, cobalt, copper, gallium, gold, hafnium, iridium, iron, lanthanum, lithium, magnesium, manganese, molybdenum, nickel, niobium, osmium, palladium, platinum, rare earth metals, rhodium, ruthenium, scandium, silver, silicon, tantalum, technetium, tin, titanium, tungsten, vanadium, yttrium, and zinc, and wherein 0.5-100% (and all values and ranges therebetween) of the rhenium content of the metal alloy is a radioactive isotope of rhenium, and wherein the metal alloy optionally includes 0-2 wt.% (and all values and ranges therebetween) of a combination of metals other than rhenium and zirconium and the metal additives, carbon, oxygen, phosphorous, sulfur, hydrogen and/or nitrogen.

In accordance with another and/or alternative aspect of the present disclosure, the metal alloy includes a) 15-50 awt.% rhenium (and all values and ranges therebetween), b) 0.5-70 awt.% molybdenum (and all values and ranges therebetween), and c) 0.1-40 wt.% (and all values and ranges therebetween) of one or more metal additives selected from aluminum, boron, beryllium, bismuth, cadmium, calcium, carbon, cerium, chromium, cobalt, copper, gallium, gold, hafnium, iridium, iron, lanthanum, lithium, magnesium, manganese, nickel, niobium, osmium, palladium, platinum, rare earth metals, rhodium, ruthenium, scandium, silver, silicon, tantalum, technetium, tin, titanium, tungsten, vanadium, yttrium, zinc, and zirconium, and wherein 0.5-100% (and all values and ranges therebetween) of the rhenium content of the metal alloy is a radioactive isotope of rhenium, and wherein the metal alloy optionally includes 0-2 wt.% (and all values and ranges therebetween) of a combination of metals other than rhenium and molybdenum and the metal additives, carbon, oxygen, phosphorous, sulfur, hydrogen and/or nitrogen.

In accordance with another and/or alternative aspect of the present disclosure, the metal alloy includes a) at least 15 awt.% rhenium, b) greater than 50 wt.% titanium (e.g., 51-80 wt.% and all values and ranges therebetween), c) 15-45 wt.% (and all values and ranges therebetween) niobium, d) 0-10 wt.% (and all values and ranges therebetween) zirconium, e) 0-15 wt.% (and all values and ranges therebetween) tantalum, and f) and 0-8 wt.% molybdenum (and all values and ranges therebetween), and wherein 0.5-100% (and all values and ranges therebetween) of the rhenium content of the metal alloy is a radioactive isotope of rhenium, and wherein the metal alloy optionally includes 0-2 wt.% (and all values and ranges therebetween) of a combination of metals other than rhenium, titanium, niobium, zirconium, tantalum, molybdenum, carbon, oxygen, phosphorous, sulfur, hydrogen and/or nitrogen.

In accordance with another and/or alternative aspect of the present disclosure, the metal alloy optionally has one or more of the following properties: i) has a yield strength of at least 110 ksi (e.g., 100-275 ksi and all values and ranges therebetween), ii) has a modulus of elasticity of at least 35000 ksi, and/or iii) a Moh’s hardness of greater than 6 (e.g., 6.05-9.8 and all values and ranges therebetween). In one non-limiting embodiment, the average Vickers hardness of the metal alloy is optionally at least about 150 Vickers (e.g., 150-300 Vickers and all values and ranges therebetween), and typically 160-240 Vickers; however, this is not required. In another and/or alternative non-limiting embodiment of the disclosure, the average ultimate tensile strength of the metal alloy is optionally at least about 125 ksi (e.g., 125-300 ksi and all values and ranges therebetween); however, this is not required. In another and/or alternative non-limiting embodiment of the disclosure, the average grain size of the metal alloy is optionally no greater than about 4 ASTM (e.g., 4 ASTM to 20 ASTM using ASTM E112 and all values and ranges therebetween, e.g., 0.35 micron to 90 micron, and all values and ranges therebetween). In another and/or alternative non-limiting embodiment of the disclosure, the average tensile elongation of the metal alloy is optionally at least about 25% (e.g., 25-50% average tensile elongation and all values and ranges therebetween).

In accordance with another and/or alternative aspect of the present disclosure, the metal alloy optionally includes a certain amount of carbon and oxygen; however, this is not required. These two elements have been found to affect the forming properties and brittleness of the metal alloy. The controlled atomic ratio of carbon and oxygen of the metal alloy also minimize the tendency of the metal alloy to form micro-cracks during the forming of the metal alloy at least partially into a desired part and/or during use of the desired part. The control of the atomic ratio of carbon to oxygen in the metal alloy allows for the redistribution of oxygen in the metal alloy to minimize the tendency of micro-cracking in the metal alloy during the forming of the metal alloy, and/or during the use of the component that is formed of or includes the metal alloy. The atomic ratio of carbon to oxygen in the metal alloy is believed to facilitate in minimizing the tendency of micro-cracking in the metal alloy and improve the degree of elongation of the metal alloy, both of which can affect one or more physical properties of the metal alloy. The carbon to oxygen atomic ratio can be as low as about 0.2:1 (e.g., 0.2:1 to 50:1 and all values and ranges therebetween). In one non-limiting formulation, the carbon to oxygen atomic ratio in the metal alloy is generally at least about 0.3:1. Typically the carbon content of the metal alloy is less than about 0.1 wt.% (e.g., 0-0.0999999 wt.% and all values and ranges therebetween), and more typically 0-0.01 wt.%. Carbon contents that are too large can adversely affect the physical properties of the metal alloy. Generally, the oxygen content is to be maintained at very low level. In one non-limiting formulation, the oxygen content is less than about 0.1 wt.% of the metal alloy (e.g., 0-0.0999999 wt.% and all values and ranges therebetween), and typically 0-0.01 wt.%. In one non-limiting arrangement, the carbon to oxygen atomic ratio in the metal alloy is at least about 2.5:1 when the oxygen content is greater than about 100 ppm in the metal alloy of the metal alloy.

In accordance with another and/or alternative aspect of the present disclosure, the metal alloy optionally includes a controlled amount of nitrogen; however, this is not required. Large amounts of nitrogen in the metal alloy can adversely affect the ductility of the metal alloy. This can in turn adversely affect the elongation properties of the metal alloy. A too high nitrogen content in the metal alloy can begin to cause the ductility of the metal alloy to unacceptably decrease, thus adversely affect one or more physical properties of the metal alloy. In one non-limiting formulation, the metal alloy includes less than about 0.001 wt.% nitrogen (e.g., 0 wt.% to 0.0009999 wt.% and all values and ranges therebetween). It is believed that the nitrogen content should be less than the content of carbon or oxygen in the metal alloy. In one non-limiting formulation, the atomic ratio of carbon to nitrogen is at least about 1.5:1 (e.g., 1.5:1 to 400:1 and all values and ranges therebetween). In another non-limiting formulation, the atomic ratio of oxygen to nitrogen is at least about 1.2:1 (e.g., 1.2:1 to 150:1 and all value and ranges therebetween).

In accordance with another and/or alternative aspect of the present disclosure, the metal alloy 1) is not clad, metal sprayed, plated and/or formed (e.g., cold worked, hot worked, etc.) onto another metal, or 2) does not have another metal or metal alloy metal sprayed, plated, clad and/or formed onto the novel metal alloy. It will be appreciated that in some applications, the novel metal alloy of the present invention may be clad, metal sprayed, plated and/or formed onto another metal, or another metal or metal alloy may be plated, metal sprayed, clad and/or formed onto the novel metal alloy when forming all or a portion of the medical device.

In accordance with another and/or alternative aspect of the present disclosure, the metal alloy is optionally used to form a coating on a portion of all of the medical device. The composition of the metal alloy coating is different from the composition of the material surface to which the metal alloy is coated. The coating thickness of the metal alloy is non-limiting (e.g., 1 µm to 1 inch and all values and ranges therebetween).

In accordance with another and/or alternative aspect of the present disclosure, the metal alloy can optionally be nitrided; however, this is not required. The nitriding process can be by gas nitriding, salt bath nitriding, or plasma nitriding. In gas nitriding, the nitrogen diffuses onto the surface of the metal alloy, thereby creating a nitrided layer. The thickness and phase constitution of the resulting nitriding layers can be selected and the process optimized for the particular properties required. The metal alloy can optionally be exposed to argon and/or hydrogen gas prior to the nitriding process to clean and/or preheat the metal alloy. These gases can be optionally used to clean oxide layers and/or solvents from the surface of the metal alloy. During the nitriding process, the metal alloy can optionally be exposed to hydrogen gas to inhibit or prevent the formation of oxides on the surface of the metal alloy. The thickness of the nitrided surface layer is less than about 1 mm. In one non-limiting embodiment, the thickness of the nitride surface layer is at least about 50 nanometer and less than about 1 mm (and all values and ranges therebetween). In another non-limiting embodiment, the thickness of the nitrided surface layer is at least about 50 nanometer and less than about 0.1 mm. Generally, the weight percent of nitrogen in the nitrided surface layer is 0.0001-5 wt.% nitrogen (and all values and ranges therebetween). The nitriding process for the metal alloy can be used to increase surface hardness and/or wear resistance of the medical devices, and/or to inhibit or prevent discoloration of the metal alloy (e.g., discoloration by oxidation, etc.).

In accordance with another and/or alternative aspect of the present disclosure, the metal alloy that is used to partially or fully form the medical device can optionally partially or fully be formed by a near net process. In one non-limiting embodiment of the disclosure, there is provided a method of metal injection molding the metal alloy to form one or more green parts of the medical device, form one or more semi-finished parts of the medical device, or form one or more finished parts of the medical device; however, it can be appreciated that the complete medical device can be formed by the method of metal injection molding the metal alloy. In another non-limiting embodiment of the disclosure, there is provided a method of powder pressing materials and optionally increasing the strength post-sintering by imparting additional cold work. In one non-limiting embodiment, the green part is pressed and then sintered. Thereafter, the sintered part is optionally again pressed to increase its mechanical strength by imparting cold work into the pressed and sintered part. Generally, the temperature during the pressing process after the sintering process is 20-100°C (and all values and ranges therebetween), typically 20-80°C, and more typically 20-40°C. As defined herein, cold working occurs at a temperature of no more than 150°C (e.g., 10-150°C and all values and ranges therebetween). The change in the shape of the repressed post-sintered part needs to be determined so the final part (pressed, sintered and re-pressed) meets the dimensional requirements of the final formed part. There is also provided a process of increasing the mechanical strength of a pressed metal part by repressing the post-sintered part to add additional cold work into the material, thereby increasing its mechanical strength. There is also provided a process of powder pressing to a green part, near net or final part using metal powder.

In accordance with another and/or alternative aspect of the present disclosure, the metal alloy that is used to partially or fully formed the medical device can be optionally formed by various techniques such as, but not limited to, 1) melting the metal alloy and/or metals that form the metal alloy (e.g., vacuum arc melting, etc.) and then extruding and/or casting the metal alloy into a portion or all of the medical device, 2) consolidating the metal powder of the metal alloy and/or metal powder of metals that form the metal alloy into a portion or all of the medical device, or 3) 3-D printing the metal powder of the metal alloy and/or metal powder of metals that form the metal alloy into a portion or all of the medical device. In one non-limiting process, the near net medical device can be formed from one or more ingots of metal or metal alloy. In one non-limiting process, an arc melting process (e.g., vacuum arc melting process, etc.) can be used to form a portion or all of the medical device, or near net component of a portion or all of the medical device. In one non-limiting embodiment, the average particle size of the metal powders is less than about 230 mesh (e.g., less than 63 microns; 1-62 microns and all values and ranges therebetween). In another and/or alternative non-limiting embodiment, the average particle size of the metal powders is about 2-62 microns, and more particularly about 5-49.9 microns. In another and/or alternative non-limiting embodiment, the average particle size of the metal powders is about 10-40 microns. In another and/or alternative non-limiting embodiment, the average density of the metal powders is greater than 5 g/cm³ (e.g., 5.001 g/cm³ to 19.3 g/cm³ and all values and ranges therebetween). In another and/or alternative non-limiting embodiment, 10-100 vol.% (and all values and ranges therebetween) of the metal powder is spherical shaped. The purity of the metal powders should be selected so that the metal powders contain very low levels of oxygen, and nitrogen. Typically, the oxygen content is less than about 50 ppm, and the nitrogen content is less than about 20 ppm. Typically, metal powder used to form the metal alloy has a purity grade of at least 99.9 and more typically at least about 99.95.

In accordance with another and/or alternative aspect of the present disclosure, when the metal alloy that is used to partially or fully formed the medical device is optionally formed by consolidated metal powder, the metal powder is optionally pressed together to form a solid solution of the metal alloy into a near net portion or all of the medical device. Typically, the pressing process is by an isostatic process (i.e., uniform pressure applied from all sides on the metal powder); however other processes can be used. When the metal powders are pressed together isostatically, cold isostatic pressing (CIP) is typically used to consolidate the metal powders; however, this is not required. The pressing process can be performed in an inert atmosphere, an oxygen-reducing atmosphere (e.g., hydrogen, argon and hydrogen mixture, etc.), and/or under a vacuum; however, this is not required. The average density of the portion or all of the medical device that is achieved by pressing together the metal powders is about 80-95% (and all values and ranges therebetween) of the final average density of the portion or all of the medical device, or about 70-96% (and all values and ranges therebetween) the minimum theoretical density of the metal alloy. Pressing pressures of at least about 300 MPa are generally used. Generally, the pressing pressure is about 400-700MPa; however, other pressures can be used. After the metal powders are pressed together, the pressed metal powders are sintered to partially or fully fuse the metal powders together to form the portion or all of the medical device. The sintering of the consolidated metal powder can be performed in an oxygen-reducing atmosphere (e.g., helium, argon, hydrogen, argon and hydrogen mixture, etc.), and/or under a vacuum; however, this is not required. At the high sintering temperatures, a high hydrogen atmosphere will reduce both the amount of carbon and oxygen in the formed portion or all of the medical device. The sintered metal powder generally has an as-sintered average density of about 90-99% the minimum theoretical density of the metal alloy. As can be appreciated, the sintering process can be used to form a coating of metal alloy on the outer surface of a substrate (e.g., metal substrate, etc.).

In accordance with another and/or alternative aspect of the present disclosure, when the metal alloy that is used to partially or fully formed the medical device is optionally partially or fully formed by a 3D printing process. In one non-limiting embodiment, the average particle size of the metal powder that is used in the 3D printing process is optionally 2-62 microns, and more particularly about 5-49.9 microns, the average density of the metal powders is greater than 5 g/cm³, and the metal powder is generally spherical-shaped, and the Hall flow (s/50 g) is less than 30 seconds (e.g., 2-29.99 seconds and all values and ranges therebetween). As can be appreciated, the 3D printing process can be used to form a coating of metal alloy on the outer surface of a substrate (e.g., metal substrate, carbide substrate, etc.).

In another and/or alternative non-limiting aspect of the present disclosure, the outer surface of the medical device or component of the medical device that is partially or fully formed of the metal alloy is optionally at least partially (e.g. 1-99.999 wt.% and all values and ranges therebetween) or fully coated with a polymer material (e.g., silicone, PTFE, ePTFE, polyurethane, polyolefins, hydrogels, biological materials [e.g., pericardium or biological polymers such as collagen, gelatin, or hyaluronic acid derivatives], etc.). The coating can be used to partially or fully encapsulate one or more portions of the medical device, and/or to partially or fully fill-in one or more of the openings in the medical device.

In another and/or alternative non-limiting aspect of the present disclosure, the outer surface of the medical device or component of the medical device that is partially or fully formed of the metal alloy is optionally at least partially (e.g. 1-99.999 wt.% and all values and ranges therebetween) or fully coated with one or more agents that facilitate in the success of the medical device and/or treated area. The term “agent” includes, but is not limited to a substance, pharmaceutical, biologic, veterinary product, drug, and analogs or derivatives otherwise formulated and/or designed to prevent, inhibit and/or treat one or more clinical and/or biological events, and/or to promote healing. One or more polymers optionally can be used to at least partially control the release of one or more agents from the medical device.

In another and/or alternative non-limiting aspect of the present disclosure, the outer surface of the medical device or component of the medical device that is partially or fully formed of the metal alloy is optionally at least partially (e.g. 1-99.999 wt.% and all values and ranges therebetween) or fully coated with an enhancement coating. As defined herein, the enhancement coat is not an agent, a polymer layer that is used with an agent, a polymer material, or a nitride coating or layer. Non-limiting enhancement coatings include chromium nitride (CrN), diamond-like carbon (DLC), titanium nitride (TiN), titanium oxynitride or titanium nitride oxide (TiNOₓ), zirconium nitride (ZrN), zirconium oxide (ZrO₂), zirconium oxynitride (ZrNₓO_{y}) [e.g., cubic ZrN:O, cubic ZrO₂:N, tetragonal ZrO₂:N, and monoclinic ZrO₂:N phase coatings], oxyzirconium-nitrogen-carbon (ZrNC), and zirconium OxyCarbide (ZrOC). The enhancement coating is used to a) reduced metal ion release of the metal material from the medical device, b) reduce the rate of corrosion on the metal that forms the medical device, c) form an outer surface of the medical device that is absent nickel and/or chromium, d) increase a surface hardness of the medical device, e) promote the formation and/or release of nitric oxide (NO) from the implanted medical device, f) promote bone healing about the medical device, g) inhibit or reduce neointimal hyperplasia/cell overgrowth onto one or more portions of the medical device after implantation in the treatment area, h) reduce infection about the medical device after implantation in the treatment area, i) reduce bacterial growth and/or bacterial infection about the implanted medical device, j) improve the biocompatibility of the implanted medical device, k) reduce the incidence of nickel exposure and/or ion release from the medical device that can react with cells about the medical device after implantation in the treatment area, and/or l) reduce inflammatory cell response about the medical device after implantation in the treatment area. In another non-limiting embodiment, the enhancement coating on one or more portions of the medical device is formulated to provide and/or promote generation of nitric oxide near, at and/or in adjacent tissue after the prosthetic valve has been expanded at the treatment site. Nitric oxide can reduce neointimal hyperplasia, reduce tissue proliferation, reduce platelet activation, reduce thrombosis, reduce restenosis, and can promote endothelial cell angiogenesis, reduce coagulation, reduce inflammation, reduce the rate of smooth muscle growth and pannus formation, all of which can contribute to an improved pro-healing environment. In another non-limiting embodiment, the enhancement coating provides, promotes and/or facilitates in a) formation or generation of nitric oxide (NO), b) stimulation of cells about the implanted medical device, and/or c) enhance modulation of cells about the implanted medical device. In one non-limiting arrangement, the enhancement coating is a metal oxynitride layer that is deposited a portion (e.g., 1-99.990% of the outer surface and all values and ranges therebetween) or all of outer surface of the medical device. In one non-limiting specific configuration, the metal oxynitride layer is or includes titanium oxynitride and/or zirconium oxynitride. In another non-limiting specific configuration, thickness of the metal oxynitride layer is 10 nanometers to 10 microns (and all values and ranges therebetween). In one non-limiting specific configuration, the oxygen to nitrogen atomic ratios of the metal oxynitride layer is 1:10 to 10:1 (and all values and ranges therebetween). In another non-limiting specific configuration, the coating of metal oxynitride layer is optionally deposited onto a metallic adhesion layer in between the base substrate of the medical device and the oxynitride layer. In another non-limiting specific configuration, the adhesion layer, when used, optionally is or includes titanium metal and/or zirconium metal. The thickness of the adhesion layer optionally has a thickness of at least 10 nanometers (e.g., 10 to 500 nanometers and all values and ranges therebetween). When the metal oxynitride layer is deposited on a portion or all of the outer surface of the medical device, the metal oxynitride coating can be used to enhance healing about the medical device and/or to increase the operational life of the medical device.

One non-limiting object of the present disclosure is the provision of metal alloy in accordance with the present disclosure that includes rhenium and is used to partially or fully form a medical device, and wherein 0.5-100% (and all values and ranges therebetween) of the rhenium content of the metal alloy is a radioactive isotope of rhenium.

Another and/or alternative non-limiting object of the present disclosure is the provision of metal alloy in accordance with the present disclosure that includes rhenium and molybdenum and is used to partially or fully form a medical device, and wherein a) 0.5-100% (and all values and ranges therebetween) of the rhenium content of the metal alloy is a radioactive isotope of rhenium, and/or b) 0-100% (and all values and ranges therebetween) of the molybdenum content of the metal alloy is a radioactive isotope of molybdenum.

Another and/or alternative non-limiting object of the present disclosure is the provision of a metal alloy that comprises rhenium, chromium, optionally molybdenum, and optionally one or more additional additives, and wherein a) 0.5-100% (and all values and ranges therebetween) of the rhenium content of the metal alloy is a radioactive isotope of rhenium, and/or b) 0-100% (and all values and ranges therebetween) of the molybdenum content of the metal alloy is a radioactive isotope of molybdenum.

Another and/or alternative non-limiting object of the present disclosure is the provision of a metal alloy that comprises at least 5 awt.% rhenium, and at least 0.1 wt.% of one or more metal additives selected from aluminum, boron, beryllium, bismuth, cadmium, calcium, carbon, cerium, chromium, cobalt, copper, gallium, gold, hafnium, iridium, iron, lanthanum, lithium, magnesium, manganese, molybdenum, nickel, niobium, osmium, palladium, platinum, rare earth metals, rhodium, ruthenium, silicon, scandium, silver, silicon, tantalum, technetium, tin, titanium, tungsten, vanadium, yttrium, zinc, and/or zirconium, and wherein 0.5-100% of the rhenium content of the metal alloy is a radioactive isotope of rhenium, and wherein 0-100% of the molybdenum content of the metal alloy, when included in the metal alloy, is a radioactive isotope of molybdenum.

Another and/or alternative non-limiting object of the present disclosure is the provision of a metal alloy that comprises rhenium in a sufficient amount to create a rhenium effect in the metal alloy, and the metal alloy is a refractory metal alloy, and wherein 0.5-100% of the rhenium content of the metal alloy is a radioactive isotope of rhenium, and wherein 0-100% of the molybdenum content of the metal alloy, when included in the metal alloy, is a radioactive isotope of molybdenum.

Another and/or alternative non-limiting object of the present disclosure is the provision of a metal alloy that comprises at least 5 awt.% rhenium in combination with stainless steel, cobalt-chromium alloy, TiAlV alloy, aluminum alloy, nickel alloy, titanium alloy, tungsten alloy, molybdenum alloy, copper alloy, beryllium-copper alloy or titanium-nickel alloy, and wherein 0.5-100% of the rhenium content of the metal alloy is a radioactive isotope of rhenium, and wherein 0-100% of the molybdenum content of the metal alloy, when included in the metal alloy, is a radioactive isotope of molybdenum.

Another and/or alternative non-limiting object of the present disclosure is the provision of medical devices that can be partially or fully formed by one or more manufacturing processes. These manufacturing processes can include, but are not limited to, laser cutting, etching, annealing, drawing, pilgering, electroplating, electro-polishing, machining, plasma coating, 3D printed coatings, 3D printing, chemical vapor deposition, chemical polishing, cleaning, pickling, ion beam deposition or implantation, sputter coating, vacuum deposition, injection molding, etc.

Another and/or alternative non-limiting object of the present disclosure is the provision of a metal alloy that is at least partially coated with an enhancement coating.

Other objects, advantages, and novel features of the present invention will become apparent from the following detailed description of the invention when considered in conjunction with the accompanying drawings.

### DETAILED DESCRIPTION OF NON-LIMITING EMBODIMENTS

A more complete understanding of the articles/devices, processes and components disclosed herein can be obtained by reference to the accompanying drawings. These figures are merely schematic representations based on convenience and the ease of demonstrating the present disclosure, and are, therefore, not intended to indicate relative size and dimensions of the devices or components thereof and/or to define or limit the scope of the exemplary embodiments.

Although specific terms are used in the following description for the sake of clarity, these terms are intended to refer only to the particular structure of the embodiments selected for illustration in the drawings and are not intended to define or limit the scope of the disclosure. In the drawings and the following description below, it is to be understood that like numeric designations refer to components of like function. The singular forms “a,” “an,” and “the” include plural referents unless the context clearly dictates otherwise.

The singular forms “a,” “an,” and “the” include plural referents unless the context clearly dictates otherwise.

As used in the specification and in the claims, the term “comprising” may include the embodiments “consisting of” and “consisting essentially of.” The terms “comprise(s),” “include(s),” “having,” “has,” “can,” “contain(s),” and variants thereof, as used herein, are intended to be open-ended transitional phrases, terms, or words that require the presence of the named ingredients/steps and permit the presence of other ingredients/steps. However, such description should be construed as also describing compositions or processes as “consisting of” and “consisting essentially of” the enumerated ingredients/steps, which allows the presence of only the named ingredients/steps, along with any unavoidable impurities that might result therefrom, and excludes other ingredients/steps.

Numerical values in the specification and claims of this application should be understood to include numerical values which are the same when reduced to the same number of significant figures and numerical values which differ from the stated value by less than the experimental error of conventional measurement technique of the type described in the present application to determine the value.

All ranges disclosed herein are inclusive of the recited endpoint and independently combinable (for example, the range of “from 2 grams to 10 grams” is inclusive of the endpoints, 2 grams and 10 grams, and all the intermediate values) and all ranges therebetween.

All ranges disclosed herein are inclusive of the recited endpoint and also include all values and ranges therebetween.

The terms “about” and “approximately” can be used to include any numerical value that can vary without changing the basic function of that value. When used with a range, “about” and “approximately” also disclose the range defined by the absolute values of the two endpoints, e.g., “about 2 to about 4” also discloses the range “from 2 to 4.” Generally, the terms “about” and “approximately” may refer to plus or minus 10% of the indicated number.

Percentages of elements should be assumed to be percent by weight of the stated element, unless expressly stated otherwise.

Additionally, the description sometimes uses terms such as “produce” and “provide” to describe the disclosed method. These terms are abstractions of the actual operations that can be performed. The actual operations that correspond to these terms can vary depending on the particular implementation and are, based on this disclosure, readily discernible by one of ordinary skill in the art.

In general, it will be apparent to one of ordinary skill in the art that at least some of the embodiments described herein can be implemented in many different embodiments of the disclosure.

The present disclosure is directed to medical devices that are partially or fully formed of a metal alloy that includes rhenium, and wherein a portion or all of the rhenium in the metal alloy is formed of radioactive isotopes of rhenium, and wherein 1) the metal alloy optionally has improved ductility as compared to the same metal alloy that is absent rhenium, 2) the metal alloy optionally has improved tensile elongation as compared to the same metal alloy that is absent rhenium, 3) the metal alloy optionally has a reduction or prevention of micro-crack formation and/or breaking of the metal alloy as compared to the same metal alloy that is absent rhenium, 4) the metal alloy optionally has improved ultimate tensile strength and/or yield strength as compared to the same metal alloy that is absent rhenium, an/or 5) the metal alloy optionally has improved hardness as compared to the same metal alloy that is absent rhenium.

The metal alloy that is used to partially or fully form the medical devices includes at least 5 awt.% rhenium, and wherein 0.5-100% (and all values and ranges therebetween) of the rhenium in the metal alloy is formed of radioactive isotopes of rhenium.

The description of embodiments and examples has been presented for purposes of illustration and description. It is not intended to be exhaustive or limiting to the forms described. Numerous modifications are possible in light of the teachings herein. Some of those modifications have been discussed, and others will be understood by those skilled in the art. The embodiments were chosen and described in order to illustrate principles of various embodiments as are suited to particular uses contemplated. The scope is, of course, not limited to the examples set forth herein, but can be employed in any number of applications and equivalent devices by those of ordinary skill in the art.

It will thus be seen that the objects set forth above, among those made apparent from the preceding description, are efficiently attained, and since certain changes may be made in the constructions set forth without departing from the spirit and scope of the invention, it is intended that all matter contained in the above description and shown in the accompanying drawings shall be interpreted as illustrative and not in a limiting sense. The invention has been described with reference to preferred and alternate embodiments. Modifications and alterations will become apparent to those skilled in the art upon reading and understanding the detailed discussion of the invention provided herein. This invention is intended to include all such modifications and alterations insofar as they come within the scope of the present invention. It is also to be understood that the following claims are intended to cover all of the generic and specific features of the invention herein described and all statements of the scope of the invention, which, as a matter of language, might be said to fall there between. The invention has been described with reference to the preferred embodiments. These and other modifications of the preferred embodiments as well as other embodiments of the invention will be obvious from the disclosure herein, whereby the foregoing descriptive matter is to be interpreted merely as illustrative of the invention and not as a limitation. It is intended to include all such modifications and alterations insofar as they come within the scope of the appended claims.

To aid the Patent Office and any readers of this application and any resulting patent in interpreting the claims appended hereto, applicants do not intend any of the appended claims or claim elements to invoke 35 U.S.C. 112(f) unless the words “means for” or “step for” are explicitly used in the particular claim.

What is claimed:

1. A metal alloy that forms a portion or all of a medical device; said metal alloy includes at least 5 awt.% rhenium and one or more alloying metals selected from the group consisting of aluminum, boron, beryllium, bismuth, cadmium, calcium, carbon, cerium, chromium, cobalt, copper, gallium, gold, hafnium, iridium, iron, lanthanum, lithium, magnesium, manganese, molybdenum and wherein said molybdenum includes 0-100% radioactive molybdenum isotopes, nickel, niobium, osmium, palladium, platinum, rare earth metals, rhodium, ruthenium, scandium, silver, silicon, tantalum, technetium, tin, titanium, tungsten, vanadium, yttrium, zinc, and zirconium; a combined weight percent of rhenium and alloying metals is at least 90 wt.% of said metal alloy; said rhenium in said metal alloy includes 0.5-100% radioactive rhenium isotopes.

2. The metal alloy as defined in claim 1, wherein said metal alloy comprises:
I) at least 15 awt.% rhenium and at least 50 wt.% iron, and one or more of a) 9-28 wt.% chromium, b) 0-35 wt.% nickel, c) 0-7 wt.% molybdenum, and wherein said molybdenum includes 0-100% radioactive molybdenum isotopes, d) 0-16 wt.% manganese, e) 0-4 wt.% silicon, f) 0-0.3 wt.% carbon, h) 0.01-1 wt.% cobalt, h) 0-5 wt.% titanium, i) 0-10 wt.% niobium, j) 0-5 wt.% copper, k) 0-4 wt.% aluminum, l) 0-10 wt. % tantalum, m) 0-1 wt.% selenium, n) 0-2 wt.% vanadium, and o) 0-2 wt.% tungsten; or
II) at least 15 awt.% rhenium and 30-68 wt.% cobalt, and 12-32 wt.% chromium, and optionally one or more of a) 0.01-38 wt.% nickel, b) 0.1-30 wt.% molybdenum, and wherein said molybdenum includes 0-100% radioactive molybdenum isotopes, c) 0-18 wt.% iron, d) 0-5 wt.% titanium, e) 0-2 wt.% manganese, f) 0-0.5 wt.% lanthanum, g) 0-0.25 wt.% carbon, h) 0-18 wt.% tungsten, i) 0-2 wt.% silicon, j) 0-2 wt.% aluminum, k) 0-2 wt.% niobium, l) 0-20 wt.% iron, m) 0-0.5 wt.% boron, n) 0-0.15 wt.% silver, and o) 0-2 wt.% titanium; or
III) at least 15 awt.% rhenium and 2-8 wt.% aluminum, and 0.01-16 wt.% vanadium, and 80-93 wt.% titanium, and optionally one or more of a) 0-1 wt.% iron, b) 0-0.2 wt.% carbon, c) 0-20 wt.% chromium, d) 0-16 wt.% molybdenum, and wherein said molybdenum includes 0-100% radioactive molybdenum isotopes, e) 0-2 wt.% nickel, f) 0-12 wt.% tin, g) 0-6 wt.% zirconium, h) 0-2 wt.% tantalum, i) 0-4 wt.% niobium, j) 0-1 wt.% silicon, k) 0-3 wt.% iron, and l) 0-0.5 wt.% yttrium.
IV) at least 15 awt.% rhenium and 70-91.5 wt.% titanium, and one or more of a) 2-8 wt.% aluminum, b) 0.01-16 wt.% vanadium, c) 0.01-1 wt.% iron, d) 0.01-0.5 wt.% yttrium, e) 0.01-20 wt.% chromium, f) 0-16 wt.% molybdenum, and wherein said molybdenum includes 0-100% radioactive molybdenum isotopes, g) 0.01-2 wt.% nickel, h) 0.01-12 wt.% tin, i) 0.01-6 wt.% zirconium, j) 0.01-2 wt.% tantalum, k) 0.01-4 wt.% niobium, l) 0.01-1 wt.% silicon, and m) 0.01-3 wt.% iron; or
V) at least 15 awt.% rhenium and 80-99 wt.% titanium, and optionally one of more of a) 0-6 wt.% aluminum, b) 0-3 wt.% tin, c) 0-1 wt.% palladium, d) 0-8 wt.% vanadium, e) 0-15 wt.% molybdenum, and wherein said molybdenum includes 0-100% radioactive molybdenum isotopes, f) 0-1 wt.% nickel, g) 0-0.3 wt.% ruthenium, h) 0-6 wt.% chromium, i) 0-4 wt.% zirconium, j) 0-4 wt.% niobium, k) 0-1 wt.% silicon, l) 0.0.5 wt.% cobalt, and m) 0-2 wt.% iron; or
VI) at least 15 awt.% rhenium and 80-99 wt.% aluminum, and optionally one or more a) 0-12 wt.% silicon, b) 0-5 wt.% magnesium, c) 0-1 wt.% manganese, d) 0-0.5 wt.% scandium, e) 0-0.5 wt.% beryllium, f) 0-0.5 wt.% yttrium, g) 0-0.5 wt.% cerium, h) 0-0.5 wt.% chromium, i) 0-3 wt.% iron, j) 0-9 wt.% zinc, j) 0-0.5 wt.% titanium, l) 0-3 wt.% lithium, m) 0-0.5 wt.% silver, n) 0-0.5 wt.% calcium, o) 0-0.5 wt.% zirconium, p) 0-1 wt.% lead, q) 0-0.5 wt.% cadmium, r) 0-0.05 wt.% bismuth, s) 0-1 wt.% nickel, t) 0-0.2 wt.% vanadium, u) 0-0.1 wt.% gallium, and v) 0-7 wt.% copper; or
VII) at least 15 awt.% rhenium and 30-98 wt.% nickel, and optionally one or more a) 5-25 wt.% chromium, b) 0-65 wt.% iron, c) 0-30 wt.% molybdenum, and wherein said molybdenum includes 0-100% radioactive molybdenum isotopes, d) 0-32 wt.% copper, e) 0-32 wt.% cobalt, f) 2-2 wt.% aluminum, g) 0-6 wt.% tantalum, h) 0-15 wt.% tungsten, i) 0-5 wt.% titanium, j) 0-6 wt.% niobium, and k) 0-3 wt.% silicon; or
VIII) at least 15 awt.% rhenium and 85-98 wt.% tungsten, and optionally one or more of a) 0-8 wt.% nickel, b) 0-5 wt.% copper, c) 0-5 wt.% molybdenum, and wherein said molybdenum includes 0-100% radioactive molybdenum isotopes, and d) 0-4 wt.% iron; or
IX) at least 15 awt.% rhenium and molybdenum alloy includes 90-99.5 wt.% molybdenum, and optionally one or more of a) 0-1 wt.% nickel, b) 0-1 wt.% titanium, c) 0-1 wt.% zirconium, d) 0-30 wt.% tungsten, e) 0-2 wt.% hafnium, f) 0-2 wt.% lanthanum; or
X) at least 15 awt.% rhenium and 55-95 wt.% copper, and optionally one or more of a) 0-40 wt.% zinc, b) 0-10 wt.% tin, c) 0-10 wt.% lead, d) 0-1 wt.% iron, e) 0-5 wt.% silicon, f) 0-12 wt.% manganese, g) 0-12 wt.% aluminum, h) 0-3 wt.% beryllium, i) 0-1 wt.% cobalt, and j) 0-20 wt.% nickel; or
XI) at least 15 awt.% rhenium and 95-98.5 wt.% copper, 1-4 wt.% beryllium, and optionally one or more of a) 0-1 wt.% cobalt, and b) 0-0.5 wt.% silicon; or
XII) at least 15 awt.% rhenium and 42-58 wt.% nickel and 42-58 wt.% titanium; or
XIII) at least 15 awt.% rhenium, and 35-84 wt.% tantalum, and optionally one or more of a) 0.1-25 wt.% tungsten, b) 0.1-30 wt.% molybdenum, and wherein said molybdenum includes 0-100% radioactive molybdenum isotopes, c) 0.01-45 wt.% niobium, d) 0.01-5 wt.% chromium, f) 0.01-5 wt.% titanium, g) 0.01-5 wt.% zirconium, and h) 0.01-4 wt.% hafnium; or
XIV) at least 15 awt.% rhenium, and 40-85 wt.% niobium, and optionally one or more of a) 0.01-20 wt.% molybdenum, and wherein said molybdenum includes 0-100% radioactive molybdenum isotopes, b) 0.01-35 wt.% tantalum, c) 0.01-12 wt.% hafnium, d) 0.01-5 wt.% zirconium, e) 0.01-3 wt.% titanium, f) 0.01-15 wt.% tungsten, and g) 0.01-1 wt.% yttrium; or
XV) at least 15 awt.% rhenium, and 30-58 wt.% titanium, and 30-58 wt.% nickel; or
XVI) at least 15 awt.% rhenium, and one or more of a) 1-85 awt.% chromium, b) 0.1-10 awt.% titanium, c) 0.1-10 awt.% molybdenum, and wherein said molybdenum includes 0-100% radioactive molybdenum isotopes, and d) 0.1-10 awt.% zirconium; or
XVII) at least 15 awt.% rhenium, and 40-93 wt.% molybdenum, and wherein said molybdenum includes 0-100% radioactive molybdenum isotopes, and one or more of a) 0.1-50 wt.% tantalum, b) 0.1-50 wt.% tungsten, c) 0.01-5 wt.% hafnium, d) 0.01-20% wt.% chromium, e) 0.01-3 wt.% titanium, and f) 0.01-2 wt.% zirconium; or
XVIII) at least 15 awt.% rhenium, and 40-85 wt.% tungsten, and one or more of a) 0.01-30 wt.% molybdenum, and wherein said molybdenum includes 0-100% radioactive molybdenum isotopes, b) 0.01-50 wt.% tantalum, d) 0.01-5 wt.% hafnium, d) 0.01-50 wt.% copper, e) 0.01-8 wt.% nickel, f) 0.01-5 wt.% iron, g) 0.01-50 wt.% zirconium, and h) 0.01-20% wt.% chromium; or
XIX) at least 15 awt.% rhenium and 0.5-70 awt.% tantalum, and 0.1-40 wt.% of one or more metal additives selected from aluminum, boron, beryllium, bismuth, cadmium, calcium, carbon, cerium, chromium, cobalt, copper, gallium, gold, hafnium, iridium, iron, lanthanum, lithium, magnesium, manganese, molybdenum and wherein said molybdenum includes 0-100% radioactive molybdenum isotopes, nickel, niobium, osmium, palladium, platinum, rare earth metals, rhodium, ruthenium, scandium, silver, silicon, technetium, tin, titanium, tungsten, vanadium, yttrium, zinc, and zirconium; or
XX) at least 15 awt.% rhenium and 0.5-70 awt.% zirconium, and 0.1-40 wt.% of one or more metal additive selected from aluminum, boron, beryllium, bismuth, cadmium, calcium, carbon, cerium, chromium, cobalt, copper, gallium, gold, hafnium, iridium, iron, lanthanum, lithium, magnesium, manganese, molybdenum and wherein said molybdenum includes 0-100% radioactive molybdenum isotopes, nickel, niobium, osmium, palladium, platinum, rare earth metals, rhodium, ruthenium, scandium, silver, silicon, tantalum, technetium, tin, titanium, tungsten, vanadium, yttrium, and zinc; or
XXI) at least 15 awt.% rhenium and 51-80 wt.% titanium, and 15-45 wt.% niobium, and optionally one or more of a) 0-10 wt.% zirconium, b) 0-15 wt.% tantalum, or c) 0-8 wt.% molybdenum, and wherein said molybdenum includes 0-100% radioactive molybdenum isotopes; or
XXII) at least 15 awt.% of said metal alloy; said metal alloy including one or more alloying metals selected from the group consisting of aluminum, boron, beryllium, bismuth, cadmium, calcium, cerium, chromium, cobalt, copper, gallium, gold, hafnium, iridium, iron, lanthanum, lithium, magnesium, manganese, 0.01-30 wt.% molybdenum, nickel, niobium, osmium, palladium, platinum, rare earth metals, rhodium, ruthenium, scandium, silver, silicon, tantalum, technetium, tin, titanium, vanadium, yttrium, zinc, and zirconium; said molybdenum in said metal alloy includes 0-100% radioactive molybdenum isotopes; a combined weight percent of said rhenium, molybdenum and said alloying metals is greater than 95 wt.%; or
XXIII) 40-60 wt.% rhenium and 40-60 wt.% molybdenum and wherein said molybdenum in said metal alloy includes 0-100% radioactive molybdenum isotopes.

3. The metal alloy as defined in claim 1, wherein said radioactive rhenium isotopes include one or more of Re-173, Re-174, Re-175, Re-176, Re-177, Re-178, Re-179, Re-180, Re-181, Re-182, Re-183, Re-184, Re-186, Re-188, Re-189, Re-190 and/or Re-191.

4. The metal alloy as defined in claim 2, wherein said radioactive rhenium isotopes include one or more of Re-173, Re-174, Re-175, Re-176, Re-177, Re-178, Re-179, Re-180, Re-181, Re-182, Re-183, Re-184, Re-186, Re-188, Re-189, Re-190 and/or Re-191.

5. The metal alloy as defined in claim 3, wherein said metal alloy include molybdenum; at least a portion of said molybdenum is formed of radioactive isotopes of molybdenum; said radioactive isotopes of molybdenum include one or more of Mo-86, Mo-87, Mo-88, Mo-89, Mo-90, Mo-91, Mo-99, Mo-101, Mo-102, Mo-103, Mo-104, Mo-105, Mo-106 and/or Mo-107.

6. The metal alloy as defined in claim 4, wherein said metal alloy include molybdenum; at least a portion of said molybdenum is formed of radioactive isotopes of molybdenum; said radioactive isotopes of molybdenum include one or more of Mo-86, Mo-87, Mo-88, Mo-89, Mo-90, Mo-91, Mo-99, Mo-101, Mo-102, Mo-103, Mo-104, Mo-105, Mo-106 and/or Mo-107.

7. The metal alloy as defined in claim 1, wherein the metal alloy is partially or fully formed by a sintering process.

8. The metal alloy as defined in claim 1, wherein said metal alloy is subjected to an injection molding process, a 3D printing process, and/or a heat and compression process to partially or fully form the medical device.

9. A medical device that is at least partially formed of a metal alloy; said metal alloy includes at least 5 awt.% rhenium and one or more alloying metals selected from the group consisting of aluminum, boron, beryllium, bismuth, cadmium, calcium, carbon, cerium, chromium, cobalt, copper, gallium, gold, hafnium, iridium, iron, lanthanum, lithium, magnesium, manganese, molybdenum and wherein said molybdenum includes 0-100% radioactive molybdenum isotopes, nickel, niobium, osmium, palladium, platinum, rare earth metals, rhodium, ruthenium, scandium, silver, silicon, tantalum, technetium, tin, titanium, tungsten, vanadium, yttrium, zinc, and zirconium; a combined weight percent of rhenium and alloying metals is at least 90 wt.% of said metal alloy; said rhenium in said metal alloy includes 0.5-100% radioactive rhenium isotopes.

10. The medical device as defined in claim 9, wherein said medical device is an orthopedic device, PFO (patent foramen ovale) device, stent, prosthetic valve, aneurysm exclusion device; LAA (left atrial appendage) closure devices, occluder, spinal implant, frame and other structures for use with a spinal implant, vascular implant, graft, staple, pacemaker, dental implant, dental crown, dental braces, wire used in medical procedures, bone implant, artificial disk, artificial spinal disk, prosthetic implant or device to repair, replace and/or support a bone, bone plate, orthopedic nail, orthopedic rod, orthopedic screw, orthopedic post, orthopedic cage, orthopedic hinge, orthopedic joint system, orthopedic anchor, orthopedic spacer, orthopedic disk, orthopedic ball, orthopedic locking connector other structural assembly.

11. The metal alloy as defined in claim 9, wherein said metal alloy comprises:
I) at least 15 awt.% rhenium and at least 50 wt.% iron, and one or more of a) 9-28 wt.% chromium, b) 0-35 wt.% nickel, c) 0-7 wt.% molybdenum, and wherein said molybdenum includes 0-100% radioactive molybdenum isotopes, d) 0-16 wt.% manganese, e) 0-4 wt.% silicon, f) 0-0.3 wt.% carbon, h) 0.01-1 wt.% cobalt, h) 0-5 wt.% titanium, i) 0-10 wt.% niobium, j) 0-5 wt.% copper, k) 0-4 wt.% aluminum, l) 0-10 wt. % tantalum, m) 0-1 wt.% selenium, n) 0-2 wt.% vanadium, and o) 0-2 wt.% tungsten; or
II) at least 15 awt.% rhenium and 30-68 wt.% cobalt, and 12-32 wt.% chromium, and optionally one or more of a) 0.01-38 wt.% nickel, b) 0.1-30 wt.% molybdenum, and wherein said molybdenum includes 0-100% radioactive molybdenum isotopes, c) 0-18 wt.% iron, d) 0-5 wt.% titanium, e) 0-2 wt.% manganese, f) 0-0.5 wt.% lanthanum, g) 0-0.25 wt.% carbon, h) 0-18 wt.% tungsten, i) 0-2 wt.% silicon, j) 0-2 wt.% aluminum, k) 0-2 wt.% niobium, l) 0-20 wt.% iron, m) 0-0.5 wt.% boron, n) 0-0.15 wt.% silver, and o) 0-2 wt.% titanium; or
III) at least 15 awt.% rhenium and 2-8 wt.% aluminum, and 0.01-16 wt.% vanadium, and 80-93 wt.% titanium, and optionally one or more of a) 0-1 wt.% iron, b) 0-0.2 wt.% carbon, c) 0-20 wt.% chromium, d) 0-16 wt.% molybdenum, and wherein said molybdenum includes 0-100% radioactive molybdenum isotopes, e) 0-2 wt.% nickel, f) 0-12 wt.% tin, g) 0-6 wt.% zirconium, h) 0-2 wt.% tantalum, i) 0-4 wt.% niobium, j) 0-1 wt.% silicon, k) 0-3 wt.% iron, and l) 0-0.5 wt.% yttrium.
IV) at least 15 awt.% rhenium and 70-91.5 wt.% titanium, and one or more of a) 2-8 wt.% aluminum, b) 0.01-16 wt.% vanadium, c) 0.01-1 wt.% iron, d) 0.01-0.5 wt.% yttrium, e) 0.01-20 wt.% chromium, f) 0-16 wt.% molybdenum, and wherein said molybdenum includes 0-100% radioactive molybdenum isotopes, g) 0.01-2 wt.% nickel, h) 0.01-12 wt.% tin, i) 0.01-6 wt.% zirconium, j) 0.01-2 wt.% tantalum, k) 0.01-4 wt.% niobium, l) 0.01-1 wt.% silicon, and m) 0.01-3 wt.% iron; or
V) at least 15 awt.% rhenium and 80-99 wt.% titanium, and optionally one of more of a) 0-6 wt.% aluminum, b) 0-3 wt.% tin, c) 0-1 wt.% palladium, d) 0-8 wt.% vanadium, e) 0-15 wt.% molybdenum, and wherein said molybdenum includes 0-100% radioactive molybdenum isotopes, f) 0-1 wt.% nickel, g) 0-0.3 wt.% ruthenium, h) 0-6 wt.% chromium, i) 0-4 wt.% zirconium, j) 0-4 wt.% niobium, k) 0-1 wt.% silicon, l) 0.0.5 wt.% cobalt, and m) 0-2 wt.% iron; or
VI) at least 15 awt.% rhenium and 80-99 wt.% aluminum, and optionally one or more a) 0-12 wt.% silicon, b) 0-5 wt.% magnesium, c) 0-1 wt.% manganese, d) 0-0.5 wt.% scandium, e) 0-0.5 wt.% beryllium, f) 0-0.5 wt.% yttrium, g) 0-0.5 wt.% cerium, h) 0-0.5 wt.% chromium, i) 0-3 wt.% iron, j) 0-9 wt.% zinc, j) 0-0.5 wt.% titanium, l) 0-3 wt.% lithium, m) 0-0.5 wt.% silver, n) 0-0.5 wt.% calcium, o) 0-0.5 wt.% zirconium, p) 0-1 wt.% lead, q) 0-0.5 wt.% cadmium, r) 0-0.05 wt.% bismuth, s) 0-1 wt.% nickel, t) 0-0.2 wt.% vanadium, u) 0-0.1 wt.% gallium, and v) 0-7 wt.% copper; or
VII) at least 15 awt.% rhenium and 30-98 wt.% nickel, and optionally one or more a) 5-25 wt.% chromium, b) 0-65 wt.% iron, c) 0-30 wt.% molybdenum, and wherein said molybdenum includes 0-100% radioactive molybdenum isotopes, d) 0-32 wt.% copper, e) 0-32 wt.% cobalt, f) 2-2 wt.% aluminum, g) 0-6 wt.% tantalum, h) 0-15 wt.% tungsten, i) 0-5 wt.% titanium, j) 0-6 wt.% niobium, and k) 0-3 wt.% silicon; or
VIII) at least 15 awt.% rhenium and 85-98 wt.% tungsten, and optionally one or more of a) 0-8 wt.% nickel, b) 0-5 wt.% copper, c) 0-5 wt.% molybdenum, and wherein said molybdenum includes 0-100% radioactive molybdenum isotopes, and d) 0-4 wt.% iron; or
IX) at least 15 awt.% rhenium and molybdenum alloy includes 90-99.5 wt.% molybdenum, and optionally one or more of a) 0-1 wt.% nickel, b) 0-1 wt.% titanium, c) 0-1 wt.% zirconium, d) 0-30 wt.% tungsten, e) 0-2 wt.% hafnium, f) 0-2 wt.% lanthanum; or
X) at least 15 awt.% rhenium and 55-95 wt.% copper, and optionally one or more of a) 0-40 wt.% zinc, b) 0-10 wt.% tin, c) 0-10 wt.% lead, d) 0-1 wt.% iron, e) 0-5 wt.% silicon, f) 0-12 wt.% manganese, g) 0-12 wt.% aluminum, h) 0-3 wt.% beryllium, i) 0-1 wt.% cobalt, and j) 0-20 wt.% nickel; or
XI) at least 15 awt.% rhenium and 95-98.5 wt.% copper, 1-4 wt.% beryllium, and optionally one or more of a) 0-1 wt.% cobalt, and b) 0-0.5 wt.% silicon; or
XII) at least 15 awt.% rhenium and 42-58 wt.% nickel and 42-58 wt.% titanium; or
XIII) at least 15 awt.% rhenium, and 35-84 wt.% tantalum, and optionally one or more of a) 0.1-25 wt.% tungsten, b) 0.1-30 wt.% molybdenum, and wherein said molybdenum includes 0-100% radioactive molybdenum isotopes, c) 0.01-45 wt.% niobium, d) 0.01-5 wt.% chromium, f) 0.01-5 wt.% titanium, g) 0.01-5 wt.% zirconium, and h) 0.01-4 wt.% hafnium; or
XIV) at least 15 awt.% rhenium, and 40-85 wt.% niobium, and optionally one or more of a) 0.01-20 wt.% molybdenum, and wherein said molybdenum includes 0-100% radioactive molybdenum isotopes, b) 0.01-35 wt.% tantalum, c) 0.01-12 wt.% hafnium, d) 0.01-5 wt.% zirconium, e) 0.01-3 wt.% titanium, f) 0.01-15 wt.% tungsten, and g) 0.01-1 wt.% yttrium; or
XV) at least 15 awt.% rhenium, and 30-58 wt.% titanium, and 30-58 wt.% nickel; or
XVI) at least 15 awt.% rhenium, and one or more of a) 1-85 awt.% chromium, b) 0.1-10 awt.% titanium, c) 0.1-10 awt.% molybdenum, and wherein said molybdenum includes 0-100% radioactive molybdenum isotopes, and d) 0.1-10 awt.% zirconium; or
XVII) at least 15 awt.% rhenium, and 40-93 wt.% molybdenum, and wherein said molybdenum includes 0-100% radioactive molybdenum isotopes, and one or more of a) 0.1-50 wt.% tantalum, b) 0.1-50 wt.% tungsten, c) 0.01-5 wt.% hafnium, d) 0.01-20% wt.% chromium, e) 0.01-3 wt.% titanium, and f) 0.01-2 wt.% zirconium; or
XVIII) at least 15 awt.% rhenium, and 40-85 wt.% tungsten, and one or more of a) 0.01-30 wt.% molybdenum, and wherein said molybdenum includes 0-100% radioactive molybdenum isotopes, b) 0.01-50 wt.% tantalum, d) 0.01-5 wt.% hafnium, d) 0.01-50 wt.% copper, e) 0.01-8 wt.% nickel, f) 0.01-5 wt.% iron, g) 0.01-50 wt.% zirconium, and h) 0.01-20% wt.% chromium; or
XIX) at least 15 awt.% rhenium and 0.5-70 awt.% tantalum, and 0.1-40 wt.% of one or more metal additives selected from aluminum, boron, beryllium, bismuth, cadmium, calcium, carbon, cerium, chromium, cobalt, copper, gallium, gold, hafnium, iridium, iron, lanthanum, lithium, magnesium, manganese, molybdenum and wherein said molybdenum includes 0-100% radioactive molybdenum isotopes, nickel, niobium, osmium, palladium, platinum, rare earth metals, rhodium, ruthenium, scandium, silver, silicon, technetium, tin, titanium, tungsten, vanadium, yttrium, zinc, and zirconium; or
XX) at least 15 awt.% rhenium and 0.5-70 awt.% zirconium, and 0.1-40 wt.% of one or more metal additive selected from aluminum, boron, beryllium, bismuth, cadmium, calcium, carbon, cerium, chromium, cobalt, copper, gallium, gold, hafnium, iridium, iron, lanthanum, lithium, magnesium, manganese, molybdenum and wherein said molybdenum includes 0-100% radioactive molybdenum isotopes, nickel, niobium, osmium, palladium, platinum, rare earth metals, rhodium, ruthenium, scandium, silver, silicon, tantalum, technetium, tin, titanium, tungsten, vanadium, yttrium, and zinc; or
XXI) at least 15 awt.% rhenium and 51-80 wt.% titanium, and 15-45 wt.% niobium, and optionally one or more of a) 0-10 wt.% zirconium, b) 0-15 wt.% tantalum, or c) 0-8 wt.% molybdenum, and wherein said molybdenum includes 0-100% radioactive molybdenum isotopes; or
XXII) at least 15 awt.% of said metal alloy; said metal alloy including one or more alloying metals selected from the group consisting of aluminum, boron, beryllium, bismuth, cadmium, calcium, cerium, chromium, cobalt, copper, gallium, gold, hafnium, iridium, iron, lanthanum, lithium, magnesium, manganese, 0.01-30 wt.% molybdenum, nickel, niobium, osmium, palladium, platinum, rare earth metals, rhodium, ruthenium, scandium, silver, silicon, tantalum, technetium, tin, titanium, vanadium, yttrium, zinc, and zirconium; said molybdenum in said metal alloy includes 0-100% radioactive molybdenum isotopes; a combined weight percent of said rhenium, molybdenum and said alloying metals is greater than 95 wt.%; or
XXIII) 40-60 wt.% rhenium and 40-60 wt.% molybdenum and wherein said molybdenum in said metal alloy includes 0-100% radioactive molybdenum isotopes.

12. The metal alloy as defined in claim 9, wherein said radioactive rhenium isotopes include one or more of Re-173, Re-174, Re-175, Re-176, Re-177, Re-178, Re-179, Re-180, Re-181, Re-182, Re-183, Re-184, Re-186, Re-188, Re-189, Re-190 and/or Re-191.

13. The metal alloy as defined in claim 11 wherein said radioactive rhenium isotopes include one or more of Re-173, Re-174, Re-175, Re-176, Re-177, Re-178, Re-179, Re-180, Re-181, Re-182, Re-183, Re-184, Re-186, Re-188, Re-189, Re-190 and/or Re-191.

14. The metal alloy as defined in claim 12, wherein said metal alloy include molybdenum; at least a portion of said molybdenum is formed of radioactive isotopes of molybdenum; said radioactive isotopes of molybdenum include one or more of Mo-86, Mo-87, Mo-88, Mo-89, Mo-90, Mo-91, Mo-99, Mo-101, Mo-102, Mo-103, Mo-104, Mo-105, Mo-106 and/or Mo-107.

15. The metal alloy as defined in claim 13, wherein said metal alloy include molybdenum; at least a portion of said molybdenum is formed of radioactive isotopes of molybdenum; said radioactive isotopes of molybdenum include one or more of Mo-86, Mo-87, Mo-88, Mo-89, Mo-90, Mo-91, Mo-99, Mo-101, Mo-102, Mo-103, Mo-104, Mo-105, Mo-106 and/or Mo-107.

16. A method for applying localized radiation to a treatment area in a patient comprising:
a. providing a medical device: medical device that is at least partially formed of a metal alloy; said metal alloy includes at least 5 awt.% rhenium and one or more alloying metals selected from the group consisting of aluminum, boron, beryllium, bismuth, cadmium, calcium, carbon, cerium, chromium, cobalt, copper, gallium, gold, hafnium, iridium, iron, lanthanum, lithium, magnesium, manganese, molybdenum and wherein said molybdenum includes 0-100% radioactive molybdenum isotopes, nickel, niobium, osmium, palladium, platinum, rare earth metals, rhodium, ruthenium, scandium, silver, silicon, tantalum, technetium, tin, titanium, tungsten, vanadium, yttrium, zinc, and zirconium; a combined weight percent of rhenium and alloying metals is at least 90 wt.% of said metal alloy; said rhenium in said metal alloy includes 0.5-100% radioactive rhenium isotopes;
b. implanting said medical device in a patient; and
wherein said radioactive rhenium isotopes from said metal alloy that at least partially forms said medical device cause localized radiation at said treatment site to a) facilitate in treatment of cancer that is located at or near said implanted medical device when implanted at said treatment site, and/or b) facilitate in treatment of cancer and/or the prevention of redevelopment of cancer at or near a location that cancerous cells currently exist and/or were removed from the patient at or near said treatment site.

17. The method as defined in claim 16, further including the step of locally inserting materials or devices that include said radioactive rhenium isotopes near or adjacent to said medical device to a) facilitate in treatment of cancer that is located at or near said implanted medical device, and/or b) facilitate in treatment of cancer and/or the prevention of redevelopment of cancer at or near a location that cancerous cells currently exist and/or were removed from the patient at or near said treatment site.

18. The method as defined in claim 16, wherein said metal alloy comprises:
I) at least 15 awt.% rhenium and at least 50 wt.% iron, and one or more of a) 9-28 wt.% chromium, b) 0-35 wt.% nickel, c) 0-7 wt.% molybdenum, and wherein said molybdenum includes 0-100% radioactive molybdenum isotopes, d) 0-16 wt.% manganese, e) 0-4 wt.% silicon, f) 0-0.3 wt.% carbon, h) 0.01-1 wt.% cobalt, h) 0-5 wt.% titanium, i) 0-10 wt.% niobium, j) 0-5 wt.% copper, k) 0-4 wt.% aluminum, l) 0-10 wt. % tantalum, m) 0-1 wt.% selenium, n) 0-2 wt.% vanadium, and o) 0-2 wt.% tungsten; or
II) at least 15 awt.% rhenium and 30-68 wt.% cobalt, and 12-32 wt.% chromium, and optionally one or more of a) 0.01-38 wt.% nickel, b) 0.1-30 wt.% molybdenum, and wherein said molybdenum includes 0-100% radioactive molybdenum isotopes, c) 0-18 wt.% iron, d) 0-5 wt.% titanium, e) 0-2 wt.% manganese, f) 0-0.5 wt.% lanthanum, g) 0-0.25 wt.% carbon, h) 0-18 wt.% tungsten, i) 0-2 wt.% silicon, j) 0-2 wt.% aluminum, k) 0-2 wt.% niobium, l) 0-20 wt.% iron, m) 0-0.5 wt.% boron, n) 0-0.15 wt.% silver, and o) 0-2 wt.% titanium; or
III) at least 15 awt.% rhenium and 2-8 wt.% aluminum, and 0.01-16 wt.% vanadium, and 80-93 wt.% titanium, and optionally one or more of a) 0-1 wt.% iron, b) 0-0.2 wt.% carbon, c) 0-20 wt.% chromium, d) 0-16 wt.% molybdenum, and wherein said molybdenum includes 0-100% radioactive molybdenum isotopes, e) 0-2 wt.% nickel, f) 0-12 wt.% tin, g) 0-6 wt.% zirconium, h) 0-2 wt.% tantalum, i) 0-4 wt.% niobium, j) 0-1 wt.% silicon, k) 0-3 wt.% iron, and l) 0-0.5 wt.% yttrium.
IV) at least 15 awt.% rhenium and 70-91.5 wt.% titanium, and one or more of a) 2-8 wt.% aluminum, b) 0.01-16 wt.% vanadium, c) 0.01-1 wt.% iron, d) 0.01-0.5 wt.% yttrium, e) 0.01-20 wt.% chromium, f) 0-16 wt.% molybdenum, and wherein said molybdenum includes 0-100% radioactive molybdenum isotopes, g) 0.01-2 wt.% nickel, h) 0.01-12 wt.% tin, i) 0.01-6 wt.% zirconium, j) 0.01-2 wt.% tantalum, k) 0.01-4 wt.% niobium, l) 0.01-1 wt.% silicon, and m) 0.01-3 wt.% iron; or
V) at least 15 awt.% rhenium and 80-99 wt.% titanium, and optionally one of more of a) 0-6 wt.% aluminum, b) 0-3 wt.% tin, c) 0-1 wt.% palladium, d) 0-8 wt.% vanadium, e) 0-15 wt.% molybdenum, and wherein said molybdenum includes 0-100% radioactive molybdenum isotopes, f) 0-1 wt.% nickel, g) 0-0.3 wt.% ruthenium, h) 0-6 wt.% chromium, i) 0-4 wt.% zirconium, j) 0-4 wt.% niobium, k) 0-1 wt.% silicon, l) 0.0.5 wt.% cobalt, and m) 0-2 wt.% iron; or
VI) at least 15 awt.% rhenium and 80-99 wt.% aluminum, and optionally one or more a) 0-12 wt.% silicon, b) 0-5 wt.% magnesium, c) 0-1 wt.% manganese, d) 0-0.5 wt.% scandium, e) 0-0.5 wt.% beryllium, f) 0-0.5 wt.% yttrium, g) 0-0.5 wt.% cerium, h) 0-0.5 wt.% chromium, i) 0-3 wt.% iron, j) 0-9 wt.% zinc, j) 0-0.5 wt.% titanium, l) 0-3 wt.% lithium, m) 0-0.5 wt.% silver, n) 0-0.5 wt.% calcium, o) 0-0.5 wt.% zirconium, p) 0-1 wt.% lead, q) 0-0.5 wt.% cadmium, r) 0-0.05 wt.% bismuth, s) 0-1 wt.% nickel, t) 0-0.2 wt.% vanadium, u) 0-0.1 wt.% gallium, and v) 0-7 wt.% copper; or
VII) at least 15 awt.% rhenium and 30-98 wt.% nickel, and optionally one or more a) 5-25 wt.% chromium, b) 0-65 wt.% iron, c) 0-30 wt.% molybdenum, and wherein said molybdenum includes 0-100% radioactive molybdenum isotopes, d) 0-32 wt.% copper, e) 0-32 wt.% cobalt, f) 2-2 wt.% aluminum, g) 0-6 wt.% tantalum, h) 0-15 wt.% tungsten, i) 0-5 wt.% titanium, j) 0-6 wt.% niobium, and k) 0-3 wt.% silicon; or
VIII) at least 15 awt.% rhenium and 85-98 wt.% tungsten, and optionally one or more of a) 0-8 wt.% nickel, b) 0-5 wt.% copper, c) 0-5 wt.% molybdenum, and wherein said molybdenum includes 0-100% radioactive molybdenum isotopes, and d) 0-4 wt.% iron; or
IX) at least 15 awt.% rhenium and molybdenum alloy includes 90-99.5 wt.% molybdenum, and optionally one or more of a) 0-1 wt.% nickel, b) 0-1 wt.% titanium, c) 0-1 wt.% zirconium, d) 0-30 wt.% tungsten, e) 0-2 wt.% hafnium, f) 0-2 wt.% lanthanum; or
X) at least 15 awt.% rhenium and 55-95 wt.% copper, and optionally one or more of a) 0-40 wt.% zinc, b) 0-10 wt.% tin, c) 0-10 wt.% lead, d) 0-1 wt.% iron, e) 0-5 wt.% silicon, f) 0-12 wt.% manganese, g) 0-12 wt.% aluminum, h) 0-3 wt.% beryllium, i) 0-1 wt.% cobalt, and j) 0-20 wt.% nickel; or
XI) at least 15 awt.% rhenium and 95-98.5 wt.% copper, 1-4 wt.% beryllium, and optionally one or more of a) 0-1 wt.% cobalt, and b) 0-0.5 wt.% silicon; or
XII) at least 15 awt.% rhenium and 42-58 wt.% nickel and 42-58 wt.% titanium; or
XIII) at least 15 awt.% rhenium, and 35-84 wt.% tantalum, and optionally one or more of a) 0.1-25 wt.% tungsten, b) 0.1-30 wt.% molybdenum, and wherein said molybdenum includes 0-100% radioactive molybdenum isotopes, c) 0.01-45 wt.% niobium, d) 0.01-5 wt.% chromium, f) 0.01-5 wt.% titanium, g) 0.01-5 wt.% zirconium, and h) 0.01-4 wt.% hafnium; or
XIV) at least 15 awt.% rhenium, and 40-85 wt.% niobium, and optionally one or more of a) 0.01-20 wt.% molybdenum, and wherein said molybdenum includes 0-100% radioactive molybdenum isotopes, b) 0.01-35 wt.% tantalum, c) 0.01-12 wt.% hafnium, d) 0.01-5 wt.% zirconium, e) 0.01-3 wt.% titanium, f) 0.01-15 wt.% tungsten, and g) 0.01-1 wt.% yttrium; or
XV) at least 15 awt.% rhenium, and 30-58 wt.% titanium, and 30-58 wt.% nickel; or
XVI) at least 15 awt.% rhenium, and one or more of a) 1-85 awt.% chromium, b) 0.1-10 awt.% titanium, c) 0.1-10 awt.% molybdenum, and wherein said molybdenum includes 0-100% radioactive molybdenum isotopes, and d) 0.1-10 awt.% zirconium; or
XVII) at least 15 awt.% rhenium, and 40-93 wt.% molybdenum, and wherein said molybdenum includes 0-100% radioactive molybdenum isotopes, and one or more of a) 0.1-50 wt.% tantalum, b) 0.1-50 wt.% tungsten, c) 0.01-5 wt.% hafnium, d) 0.01-20% wt.% chromium, e) 0.01-3 wt.% titanium, and f) 0.01-2 wt.% zirconium; or
XVIII) at least 15 awt.% rhenium, and 40-85 wt.% tungsten, and one or more of a) 0.01-30 wt.% molybdenum, and wherein said molybdenum includes 0-100% radioactive molybdenum isotopes, b) 0.01-50 wt.% tantalum, d) 0.01-5 wt.% hafnium, d) 0.01-50 wt.% copper, e) 0.01-8 wt.% nickel, f) 0.01-5 wt.% iron, g) 0.01-50 wt.% zirconium, and h) 0.01-20% wt.% chromium; or
XIX) at least 15 awt.% rhenium and 0.5-70 awt.% tantalum, and 0.1-40 wt.% of one or more metal additives selected from aluminum, boron, beryllium, bismuth, cadmium, calcium, carbon, cerium, chromium, cobalt, copper, gallium, gold, hafnium, iridium, iron, lanthanum, lithium, magnesium, manganese, molybdenum and wherein said molybdenum includes 0-100% radioactive molybdenum isotopes, nickel, niobium, osmium, palladium, platinum, rare earth metals, rhodium, ruthenium, scandium, silver, silicon, technetium, tin, titanium, tungsten, vanadium, yttrium, zinc, and zirconium; or
XX) at least 15 awt.% rhenium and 0.5-70 awt.% zirconium, and 0.1-40 wt.% of one or more metal additive selected from aluminum, boron, beryllium, bismuth, cadmium, calcium, carbon, cerium, chromium, cobalt, copper, gallium, gold, hafnium, iridium, iron, lanthanum, lithium, magnesium, manganese, molybdenum and wherein said molybdenum includes 0-100% radioactive molybdenum isotopes, nickel, niobium, osmium, palladium, platinum, rare earth metals, rhodium, ruthenium, scandium, silver, silicon, tantalum, technetium, tin, titanium, tungsten, vanadium, yttrium, and zinc; or
XXI) at least 15 awt.% rhenium and 51-80 wt.% titanium, and 15-45 wt.% niobium, and optionally one or more of a) 0-10 wt.% zirconium, b) 0-15 wt.% tantalum, or c) 0-8 wt.% molybdenum, and wherein said molybdenum includes 0-100% radioactive molybdenum isotopes; or
XXII) at least 15 awt.% of said metal alloy; said metal alloy including one or more alloying metals selected from the group consisting of aluminum, boron, beryllium, bismuth, cadmium, calcium, cerium, chromium, cobalt, copper, gallium, gold, hafnium, iridium, iron, lanthanum, lithium, magnesium, manganese, 0.01-30 wt.% molybdenum, nickel, niobium, osmium, palladium, platinum, rare earth metals, rhodium, ruthenium, scandium, silver, silicon, tantalum, technetium, tin, titanium, vanadium, yttrium, zinc, and zirconium; said molybdenum in said metal alloy includes 0-100% radioactive molybdenum isotopes; a combined weight percent of said rhenium, molybdenum and said alloying metals is greater than 95 wt.%; or
XXIII) 40-60 wt.% rhenium and 40-60 wt.% molybdenum and wherein said molybdenum in said metal alloy includes 0-100% radioactive molybdenum isotopes.

19. The method as defined in claim 18, wherein said radioactive rhenium isotopes include one or more of Re-173, Re-174, Re-175, Re-176, Re-177, Re-178, Re-179, Re-180, Re-181, Re-182, Re-183, Re-184, Re-186, Re-188, Re-189, Re-190 and/or Re-191.

20. The method as defined in claim 19, wherein said metal alloy include molybdenum; at least a portion of said molybdenum is formed of radioactive isotopes of molybdenum; said radioactive isotopes of molybdenum include one or more of Mo-86, Mo-87, Mo-88, Mo-89, Mo-90, Mo-91, Mo-99, Mo-101, Mo-102, Mo-103, Mo-104, Mo-105, Mo-106 and/or Mo-107.
